(19) United States
(12) Patent Application Publication
(10) Pub. No.: US 2023/0398243 A1
(43) Pub. Date: Dec. 14, 2023

(54) THERAPEUTIC, RADIOLABELED NANOPARTICLES AND METHODS OF USE THEREOF

(71) Applicant: The General Hospital Corporation, Boston, MA (US)

(72) Inventors: Zdravka Medarova, Methuen, MA (US); Sergey Shuvaev, Boston, MA (US); Peter Caravan, Charlestown, MA (US); Mariane Le Fur, Boston, MA (US); Byunghee Yoo, Lexington, MA (US)

(21) Appl. No.: 18/035,174

(22) PCT Filed: Nov. 3, 2021
(86) PCT No.: PCT/US2021/057912

§ 371 Date: May 3, 2023

Related U.S. Application Data
(60) Provisional application No. 63/109,298, filed on Nov. 3, 2020.

(51) Int. Cl. A61K 51/12; A61P 35/04; A61K 51/04

(52) U.S. Cl. CPC A61K 51/1244; A61P 35/04; A61K 51/0491; A61K 51/0482; B82Y 5/00

(57) ABSTRACT

Provided herein are therapeutic nanoparticles including a radiolabel, a chelator that is covalently linked to the therapeutic nanoparticle and to the radiolabel, and a nucleic acid molecule that is covalently linked to the therapeutic nanoparticle. The therapeutic nanoparticle has a diameter between about 10 nanometers (nm) to about 30 nm, and the therapeutic nanoparticle is magnetic. Also provided are pharmaceutical compositions containing these therapeutic nanoparticles. Also provided herein are methods of decreasing cancer cell invasion or metastasis in a subject having a cancer and methods of treating a metastatic cancer in a lymph node in a subject that require the administration of these therapeutic nanoparticles to a subject. Also provided herein are methods of detecting, diagnosing, and/or monitoring a metastatic cancer tissue in a subject. Also provided herein are methods of preparing these therapeutic nanoparticles.

### CROSS-REFERENCE TO RELATED APPLICATIONS

This application claims the benefit of U.S. Provisional Application No. 63/109,298, filed on Nov. 3, 2020. The entire contents of the foregoing are incorporated herein by reference in their entireties.

### FEDERALLY SPONSORED RESEARCH OR DEVELOPMENT

This invention was made with Government support under Grant Number CA16346101 awarded by the National Cancer Institute of the National Institutes of Health. The Government has certain rights in the invention.

### TECHNICAL FIELD

This disclosure relates to therapeutic, radiolabeled nanoparticles including a polymer coating and a covalently-linked inhibitory nucleic acid, compositions containing these therapeutic nanoparticles, methods of using these therapeutic nanoparticles, and methods of preparing these therapeutic nanoparticles.

### BACKGROUND

Conventional therapies targeted towards primary tumor cells oftentimes do not affect the metastatic cells and, in fact, may promote metastasis. This explains the poor outcomes in patients diagnosed with metastatic disease despite the good prognosis of patients with localized cancer of the same organ of origin (Steeg P.S. Nat. Rev. Cancer. 2016; 16:201-218). For these reasons, there is a need for therapies specific to unique properties of metastatic tumor cells. These cells have the ability to break out of the primary tumor mass, travel through the circulation, and colonize a new vital organ in the process of metastasis. Importantly, these cells are genetically and phenotypically distinct from the majority of the cells in the tumor mass, spawning metastatic lesions that have diverged in their gene expression profile from their respective primary tumors.

### SUMMARY

Certain aspects of the present disclosure are directed to a therapeutic nanoparticle including a radiolabel; a chelator that is covalently linked to the therapeutic nanoparticle and to the radiolabel; and a nucleic acid molecule that is covalently linked to the therapeutic nanoparticle, wherein the therapeutic nanoparticle has a diameter between about 10 nanometers (nm) to about 30 nm, and wherein the therapeutic nanoparticle is magnetic.

In some embodiments, the chelator is covalently-linked to the therapeutic nanoparticle through a chemical moiety comprising a secondary amine. In some embodiments, the chelator comprises 1,4,7-triazacyclononane,1-glutaric acid-4,7-acetic acid (NODAGA). In some embodiments, the chelator comprises DOTA, DOTA-GA, p-SCN-Bn-DOTA, CB-IE2A, CB-IE1A1P, AAZTA, MeCOSar, p-SCN-Bn-NOTA, NOTA, HBED-CC, THP, MASS, DFO, or any combination thereof. In some embodiments, the radiolabel comprises copper-64 (Cu-64). In some embodiments, the radiolabel comprises copper-67 (Cu-67), yttrium-90 (Y-90), terbium-161 (Tb-161), lutetium-177 (Lu-177), bismuth-231(Bi-213), lead-212 (Pb-212), actinium-225 (Ac-225), zirconium-89 (Zr), or any combination thereof. In some embodiments, the nucleic acid molecule comprises at least one modified nucleotide. In some embodiments, the at least one modified nucleotide is a locked nucleotide. In some embodiments, the nucleic acid molecule is an antagomir. In some embodiments, the antagomir inhibits microRNA-10b (miR-10b). In some embodiments, the nucleic acid molecule is covalently-linked to the nanoparticle through a chemical moiety comprising a disulfide bond. In some embodiments, the therapeutic nanoparticle comprises an iron oxide core. In some embodiments, the nanoparticle further comprises a polymer coating. In some embodiments, the polymer coating comprises dextran.

In another aspect, the present disclosure is directed to a pharmaceutical composition including any of the therapeutic nanoparticle discloses herein. In some embodiments, the pharmaceutical composition further includes at least one pharmaceutically acceptable carrier or diluent. In some embodiments, the pharmaceutical composition is formulated into a dosage form that is an injectable, a tablet, a lyophilized powder, a suspension, or any combination thereof.

In another aspect, the present disclosure is directed to a method for decreasing cancer cell invasion or metastasis in a subject having a cancer, the method comprising administering any of the therapeutic nanoparticles disclosed herein to the subject having the cancer, wherein the therapeutic nanoparticle is administered in an amount sufficient to decrease cancer cell invasion or metastasis in the subject.

In some embodiments, the therapeutic nanoparticle is administered to the subject at a dose that is less than about 0.014 mg/kg. In some embodiments, the cancer cell metastasis is from a primary tumor to a lymph node in the subject or is from a lymph node to a secondary tissue in a subject. In some embodiments, the cancer cell is selected from the group consisting of: a breast cancer cell, a colon cancer cell, a kidney cancer cell, a lung cancer cell, a skin cancer cell, an ovarian cancer cell, a pancreatic cancer cell, a prostate cancer cell, a rectal cancer cell, a stomach cancer cell, a thyroid cancer cell, and a uterine cancer cell. In some embodiments, the method further includes imaging a tissue of the subject to determine the location or number of cancer cells in the subject, or the location of the therapeutic nanoparticles in the subject.

In another aspect, the present disclosure is directed to a method for treating a metastatic cancer in a lymph node in a subject, the method comprising administering any of the therapeutic nanoparticles disclosed herein to a lymph node of a subject having a metastatic cancer, wherein the therapeutic nanoparticle is administered in an amount sufficient to treat the metastatic cancer in the lymph node in the subject.

In some embodiments, the metastatic cancer results from a primary breast cancer. In some embodiments, the administering results in a decrease or stabilization of metastatic tumor size, or a decrease in the rate of metastatic tumor growth in a lymph node in the subject.

In another aspect, the present disclosure is directed to a method for detecting, diagnosing, and/or monitoring a metastatic cancer tissue in a subject. The methods includes administering any of the therapeutic nanoparticles disclosed herein to the subject having the metastatic cancer tissue; and imaging the therapeutic nanoparticle, wherein the therapeutic nanoparticle is administered in an amount sufficient to image the therapeutic nanoparticle in the subject.

In some embodiments, the imaging is carried out by magnetic resonance imaging (MRI), positron emission tomography (PET), single-photon emission computed tomography (SPECT), computer tomography (CT), or any combination thereof. In some embodiments, the therapeutic nanoparticle is administered in two or more doses to the subject. In some embodiments, the therapeutic nanoparticle is administered to the subject at least once a week. In some embodiments, the therapeutic nanoparticle is administered to the subject by intravenous, subcutaneous, intraarterial, intramuscular, or intraperitoneal administration. In some embodiments, the subject is further administered a chemotherapeutic agent.

In another aspect, the present disclosure is directed to a method for preparing any of the therapeutic nanoparticles discloses herein, the method including: preparing the magnetic nanoparticle; covalently linking the nucleic acid molecule to the magnetic nanoparticle; covalently linking the chelator to the magnetic nanoparticle by reacting the magnetic nanoparticle with the chelator at a ratio of about 40 chelator equivalents per magnetic nanoparticle; adding a solution of ⁶⁴CuCl₂ to the magnetic nanoparticle; and purifying a mixture of the solution of ⁶⁴CuCl₂ and the magnetic nanoparticle to yield the therapeutic nanoparticle.

In some embodiments, covalently linking the chelator to the magnetic nanoparticle is performed at a temperature of about 0° C. to about 8° C. In some embodiments, the method further includes heating the mixture of the solution of ⁶⁴CuCl₂ and the magnetic nanoparticle at a temperature of about 40° C. to about 65° C. In some embodiments, the mixture is heated for about 10 minutes to about 30 minutes.

The term “magnetic” is used to describe a composition that is responsive to a magnetic field. Non-limiting examples of magnetic compositions (e.g., any of the nanoparticle compositions described herein) can contain a material that is paramagnetic, superparamagnetic, ferromagnetic, or diamagnetic. Non-limiting examples of magnetic compositions contain a metal oxide selected from the group of: magnetite; ferrites (e.g., ferrites of manganese, cobalt, and nickel); Fe(II) oxides; and hematite, and metal alloys thereof. Additional magnetic materials are described herein and are known in the art.

The term “diamagnetic” is used to describe a composition that has a relative magnetic permeability that is less than or equal to 1 and that is repelled by a magnetic field.

The term “paramagnetic” is used to describe a composition that develops a magnetic moment only in the presence of an externally applied magnetic field.

The term “ferromagnetic” or “ferromagnetic” is used to describe a composition that is strongly susceptible to magnetic fields and is capable of retaining magnetic properties (a magnetic moment) after an externally applied magnetic field has been removed.

By the term “nanoparticle” is meant an object that has a diameter between about 2 nm to about 200 nm (e.g., between 10 nm and 200 nm, between 2 nm and 100 nm, between 2 nm and 40 nm, between 2 nm and 30 nm, between 2 nm and 20 nm, between 2 nm and 15 nm, between 100 nm and 200 nm, and between 150 nm and 200 nm). Non-limiting examples of nanoparticles include the nanoparticles described herein.

By the term “magnetic nanoparticle” is meant a nanoparticle (e.g., any of the nanoparticles described herein) that is magnetic (as defined herein). Non-limiting examples of magnetic nanoparticles are described herein. Additional magnetic nanoparticles are known in the art.

By the term “nucleic acid” is meant any single- or double-stranded polynucleotide (e.g., DNA or RNA, cDNA, semi-synthetic, or synthetic origin). The term nucleic acid includes oligonucleotides containing at least one modified nucleotide (e.g., containing a modification in the base and/or a modification in the sugar) and/or a modification in the phosphodiester bond linking two nucleotides. In some embodiments, the nucleic acid can contain at least one locked nucleotide (LNA). Non-limiting examples of nucleic acids are described herein. Additional examples of nucleic acids are known in the art.

By the term “modified nucleotide” is meant a DNA or RNA nucleotide that contains at least one modification in its base and/or at least one modification in its sugar (ribose or deoxyribose). A modified nucleotide can also contain modification in an atom that forms a phosphodiester bond between two adjoining nucleotides in a nucleic acid sequence.

By the term “polymer coating” is meant at least one molecular layer (e.g., homogenous or non-homogenous) of at least one polymer (e.g., dextran) applied to a surface of a three-dimensional object (e.g., a three-dimensional object containing a magnetic material, such as a metal oxide). Non-limiting examples of polymers that can be used to generate a polymer coating are described herein. Additional examples of polymers that can be used to generate a polymer coating are known in the art. Methods for applying a polymer coating to an object (e.g., a three-dimensional object containing a magnetic material) are described herein and are also known in the art.

By the phrase “cancer cell invasion” is meant the migration of a cancer cell into a non-cancerous tissue in a subject. Non-limiting examples of cancer cell invasion include: the migration of a cancer cell into a lymph node, the lymph, the vasculature (e.g., adventitia, media, or intima of a blood vessel), or an epithelial or endothelial tissue. Exemplary methods for detecting and determining cancer cell invasion are described herein. Additional methods for detecting and determining cancer cell invasion are known in the art.

By the term “metastasis” is meant the migration of a cancer cell present in a primary tumor to a secondary, non-adjacent tissue in a subject. Non-limiting examples of metastasis include: metastasis from a primary tumor to a lymph node (e.g., a regional lymph node), bone tissue, lung tissue, liver tissue, and/or brain tissue. The term metastasis also includes the migration of a metastatic cancer cell found in a lymph node to a secondary tissue (e.g., bone tissue, liver tissue, or brain tissue). In some non-limiting embodiments, the cancer cell present in a primary tumor is a breast cancer cell, a colon cancer cell, a kidney cancer cell, a lung cancer cell, a skin cancer cell, an ovarian cancer cell, a pancreatic cancer cell, a prostate cancer cell, a rectal cancer cell, a stomach cancer cell, a thyroid cancer cell, or a uterine cancer cell. Additional aspects and examples of metastasis are known in the art or described herein.

By the term “primary tumor” is meant a tumor present at the anatomical site where tumor progression began and proceeded to yield a cancerous mass. In some embodiments, a physician may not be able to clearly identify the site of the primary tumor in a subject.

By the term “metastatic tumor” is meant a tumor in a subject that originated from a tumor cell that metastasized from a primary tumor in the subject. In some embodiments, a physician may not be able to clearly identify the site of the primary tumor in a subject.

By the term “lymph node” is meant a small spherical or oval-shaped organ of the immune system that contains a variety of cells including B-lymphocytes, T-lymphocytes, and macrophages, which is connected to the lymphatic system by lymph vessels. A variety of lymph nodes are present in a mammal including, but not limited to: axillary lymph nodes (e.g., lateral glands, anterior or pectoral glands, posterior or subscapular glands, central or intermediate glands, or medial or subclavicular glands), sentinel lymph nodes, sub-mandibular lymph nodes, anterior cervical lymph nodes, posterior cervical lymph nodes, supraclavicular lymph nodes, sub-mental lymph nodes, femoral lymph nodes, mesenteric lymph nodes, mediastinal lymph nodes, inguinal lymph nodes, subsegmental lymph nodes, segmental lymph nodes, lobar lymph nodes, interlobar lymph nodes, hilar lymph nodes, supratrochlear glands, deltoideopectoral glands, superficial inguinal lymph nodes, deep inguinal lymph nodes, brachial lymph nodes, and popliteal lymph nodes.

By the term “imaging” is meant the visualization of at least one tissue of a subject using a biophysical technique (e.g., electromagnetic energy absorption and/or emission). Non-limiting embodiments of imaging include magnetic resonance imaging (MRI), X-ray computed tomography, and optical imaging.

The terms “subject” or “patient,” as used herein, refer to any mammal (e.g., a human or a veterinary subject, e.g., a dog, cat, horse, cow, goat, sheep, mouse, rat, or rabbit) to which a composition or method of the present disclosure may be administered, e.g., for experimental, diagnostic, prophylactic, and/or therapeutic purposes. The subject may seek or need treatment, require treatment, is receiving treatment, will receive treatment, or is under care by a trained professional for a particular disease or condition.

As used in the specification and the appended claims, the singular forms “a,” “an” and “the” include plural referents unless the context clearly dictates otherwise. Thus, for example, reference to “a nanoparticle” includes mixtures of nanoparticles, reference to “a nanoparticle” includes mixtures of two or more such nanoparticles, and the like.

Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

By the term “chemotherapeutic agent” is meant a molecule that can be used to reduce the rate of cancer cell growth or to induce or mediate the death (e.g., necrosis or apoptosis) of cancer cells in a subject (e.g., a human). In non-limiting examples, a chemotherapeutic agent can be a small molecule, a protein (e.g., an antibody, an antigen-binding fragment of an antibody, or a derivative or conjugate thereof), a nucleic acid, or any combination thereof. Non-limiting examples of chemotherapeutic agents include: cyclophosphamide, mechlorethamine, chlorabucil, melphalan, daunorubicin, doxorubicin, epirubicin, idarubicin, mitoxantrone, valrubicin, paclitaxel, docetaxel, etoposide, teniposide, tafluposide, azacitidine, axathioprine, capecitabine, cytarabine, doxifluridine, fluorouracil, gemcitabine, mercaptopurine, methotrexate, tioguanine, bleomycin, carboplatin, cisplatin, oxaliplatin, all-trans retinoic acid, vinblastine, vincristine, vindesine, vinorelbine, and bevacizumab (or an antigen-binding fragment thereof). Additional examples of chemotherapeutic agents are known in the art.

Embodiments disclosed below include therapeutic, radiolabeled nanoparticles including a polymer coating and a covalently-linked inhibitory nucleic acid, compositions containing these therapeutic nanoparticles, methods of using these therapeutic nanoparticles, and methods of preparing these therapeutic nanoparticles. Some embodiments of the therapeutic nanoparticles, compositions, and methods described herein may provide one or more of the following advantages.

First, certain embodiments of the present disclosure include methods of using any of the nanoparticle compositions for the treatment, prevention, diagnosing, and/or imaging of a disease (e.g., cancer) in a subject in need thereof. There is currently a need for improved therapeutics that can significantly accumulate in metastatic tissues for treatment and/or diagnostic purposes. The therapeutic nanoparticles, compositions and methods of the present disclosure address this need. For example, in some embodiments, the therapeutic nanoparticles described herein can accumulate in metastatic tissues. In some embodiments, the therapeutic nanoparticles can include a radiolabel that enables imaging (e.g., via positron emission tomography (PET)) of the therapeutic nanoparticles. In some embodiments, the therapeutic nanoparticles can further include an inhibitory nucleic acid that may cause complete and persistent regression of metastases. Thus, in some embodiments, the therapeutic nanoparticles of the disclosure can effectively target metastatic tissues.

Second, some embodiments described herein may provide enough sensitivity (e.g., using PET as the imaging technique) to determine the concentration of radiolabeled drug with sensitivity approaching the sub-picomolar range. Thus, in some embodiments, the therapeutic nanoparticles can advantageously be detected and/or imaged while administering a dose as little as one microgram. This characteristic has significant advantages in the initial phases of drug development. Because such a low dose does not induce adverse side effects, approval from the U.S. Food and Drug Administration for initial human studies may be obtained more quickly and with a more limited preclinical safety and toxicology dossier than is required for therapeutic agents.

Third, some embodiments described herein may clarify the biodistribution of the therapeutic nanoparticles in cancer patients. One of the major challenges facing the development of cancer therapeutics lies in the effective delivery to the target organs. In the case of drug delivery to metastases, complicating factors include the larger size of the lesions, as compared to animal models, the heterogeneity of human disease, and differences in the pharmacokinetics of the drugs, due to interspecies hemodynamic variability. Based on these differences, it is not possible to directly extrapolate proof of successful clinical implementation of therapeutic agents from preclinical biodistribution and efficacy data. Based on the comparable biodistribution of the therapeutic microdose to the therapeutic macrodose (see e.g., FIGS. 11A-11D), certain embodiments described herein may reveal the pharmacokinetic behavior of the therapeutic nanoparticles without the need to administer doses greater than about 0.014 mg/kg, may help establish dosing during therapy, and may enable selecting patients for treatment based on which patients' metastases accumulate the therapeutic nanoparticles.

Where values are described in the present disclosure in terms of ranges, endpoints are included. Furthermore, it should be understood that the description includes the disclosure of all possible sub-ranges within such ranges, as well as specific numerical values that fall within such ranges irrespective of whether a specific numerical value or specific sub-range is expressly stated.

Various embodiments of the features of this disclosure are described herein. However, it should be understood that such embodiments are provided merely by way of example, and numerous variations, changes, and substitutions can occur according to those skilled in the art without departing from the scope of this disclosure. It should also be understood that various alternatives to the specific embodiments described herein are also within the scope of this disclosure.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

The details of one or more embodiments of the invention are set forth in the accompanying drawings and the description below. Other features and advantages of the invention will be apparent from the following detailed description, figures, and claims.

### DESCRIPTION OF DRAWINGS

FIG. 1A is a diagram showing an exemplary therapeutic magnetic nanoparticle (MN-anti-miR10b) having a dextran-coating and an antisense LNA oligonucleotide targeting miRNA-10b. FIG. 1B is a graph showing the levels of miR-10b expression determined by quantitative reverse transcriptase polymerase chain reaction (qRT-PCR) in human metastatic breast cancer cells following a 48-h incubation of with MN-anti-miR10b or a corresponding magnetic nanoparticle (MN-scr-miR) containing a scrambled nucleic acid rather than the anti-miR10b nucleic acid. The data shown are represented as mean±standard deviation (n=3; **, p<0.01).

FIG. 2A is a set of six photomicrographs showing the cell morphology of human metastatic breast cancer cells 48 hr following treatment with phosphate buffer saline (PBS) (negative control), a low dose of doxorubicin alone (dox), magnetic nanoparticles containing a scrambled nucleic acid rather than the anti-miR10b nucleic acid (MN-scr-miR), magnetic nanoparticles containing a scrambled nucleic acid and a low dose of doxorubicin (MN-scr-miR+dox), MN-anti-miR10b, and MN-anti-miR10b and a low-dose doxorubicin (MN-anti-miR10b+dox). FIG. 2B is a graph showing the percent of apoptotic cells for each of the aforementioned conditions. FIG. 2C is a graph showing the percent of cell proliferation for each of the aforementioned conditions. FIG. 2D is a set of six graphs of flow cytometry data of human metastatic breast cancer cells showing pre-G₁ apoptotic and G₂/M arrested cell populations for each of the aforementioned conditions. (Data represent average±standard deviation; two-tailed t-test; n=3).

FIG. 3A is an image of gel electrophoresis confirming miR-10b knock-out. FIG. 3B is a set of six photomicrographs of tumor cells taken 24 hrs and 72 hrs after transfection with a knock-out vector set (TALENs L+R).

FIG. 4A is a set of seven macroscopic images generated using bioluminescence imaging (BLI) and a set of seven macroscopic images generated using near infrared fluorescence (NIRF) imaging of excised organs with metastases showing accumulation of MN-anti-miR10b. FIG. 4B is a set of a set of seven photomicrographs of excised organs stained with hematoxylin and eosin (H&E) and a set of a set of seven photomicrographs of excised organs fluorescently stained with Cy5.5. FIG. 4C shows BLI and NIRF images of MN-anti-miR10b delivery to brain metastases in vivo on the left; on the right, three fluorescence microscopy photomicrographs show the accumulation of MN-anti-miR10b (Cy5.5 on MN) in luciferase-expressing tumor cells.

FIG. 5A is a set of bioluminescence images of metastatic burden in animals treated with PBS (negative control), magnetic nanoparticles containing a scrambled nucleic acid rather than the anti-miR10b nucleic acid (MN-scr-miR), magnetic nanoparticles containing a scrambled nucleic acid and a low dose of doxorubicin (MN-scr-miR+dox), MN-anti-miR10b, and MN-anti-miR10b and a low-dose doxorubicin (MN-anti-miR10b+dox). FIG. 5B is a graph showing a quantitative analysis of metastatic burden from all treatment groups indicating complete regression of metastatic burden in the lymph nodes of experimental animals after just 4 weekly treatments. Background counts are derived from non-tumor bearing animals. FIG. 5C is a set of bioluminescence images and photographs of ex vivo organs showing the absence of detectable lymph node or lung metastases in mice treated with MN-anti-miR10b+dox. Control treatments included PBS, MN-scr-miR, MN-scr-miR+dox, and MN-anti-miR10b. FIG. 5D is a graph showing the body weight of the mice throughout the time course of the study. FIG. 5E is a graph showing the survival probability of the mice treated with PBS, MN-scr-miR, MN-scr-miR+dox, MN-anti-miR10b, and MN-anti-miR10b+dox. (Data represent average±standard error of the mean; within-Subjects ANOVA: p<0.05. PBS, n=2; MN-scr-miR, n=6; MN-scrmiR+dox, n=10; MN-anti-miR10b, n=7; MN-anti-miR10b+dox, n=10).

FIG. 6A is a set of bioluminescence images of the mice showing metastatic burden. FIG. 6B is a graph showing a quantitative analysis of relative metastatic burden in all treatment groups. FIG. 6C is a graph showing the fraction of survival of the mice treated PBS, HD Dox, MN-scr-miR+dox, and MN-anti-miR10b+dox. FIG. 6D is a set of three images of the lungs post-necropsy showing no evidence of lung metastases in the animals treated with MN-anti-miR10b.

FIG. 7 is a schematic depicting the Preparation of ^{nat/64}Cu-MN-anti-miR10b: Step 1. Coupling reaction between MN-NH2 and NODAGA-NHS to form NODAGA-MN. Step 2. Functionalization with the hetero-bifunctional linker, SPDP. Step 3. Conjugation to anti-miR10b antagomir via disulfide linkage to form NODAGA-MN-anti-miR10b. Step 4. Complexation reaction with ^{nat}CuCl₂ or ⁶⁴CuCl₂ leading to ^{nat/64}Cu-MN-anti-miR10b.

FIG. 8A is a graph showing radiochemical purity confirmed by iTLC; (top) unlabeled Cu-64, (middle) separation of ⁶⁴Cu-MN-anti-miR10b and unlabeled Cu-64, and (bottom) 64Cu-MN-anti-miR10b after PD-10 purification. FIG. 8B shows high-performance liquid chromatography (HPLC) traces of 64 Cu-MN-anti-miR10b using size exclusion chromatography (top) and UV trace at 254 nm (bottom). FIG. 8C is a set of two transmission electron microscopy (TEM) images of Cu-MN-anti-miR10b and natCu-MN-anti-miR10b. FIG. 8D is a graph showing nanoparticle size as characterized by TEM and dynamic light scattering (DLS). FIG. 8E is a graph showing in vitro cell uptake by breast adenocarcinoma cells. FIG. 8F is a graph showing the levels of miR-relative expression determined by qRT-PCR demonstrating target engagement (inhibition of miR-10b). ^{nat}Cu was utilized for the preparation of natCu-MN-anti-miR10b (t-test, n=3, **P<0.01).

FIG. 9 is a schematic exemplary synthetic pathways for radiolabeling of MN-anti-miR10b nanoparticles.

FIG. 10 is a set of exemplary chelators that can be used to prepare the magnetic, radiolabeled nanoparticles of the disclosure.

FIG. 11A is a graph showing the ex vivo biodistribution measured at 24 and 48 h after administration of a microdose of ⁶⁴Cu-MN-anti-miR10b (% ID/g). FIG. 11B is a graph showing the ex vivo biodistribution measured at 24 and 48 h after administration of a therapeutic carrier-added macrodose of ⁶⁴Cu-MN-anti-miR10b (% ID/g). Insets show the % ID/g values in liver and spleen. ^{#}Denotes organs with metastasis as detected by BLI.

Error bars represent standard error of the mean. FIGS. 11C and 11D are graphs showing the correlation between the % ID/g in non-metastatic organs obtained after administration of a microdose and the % ID/g obtained after administration of a macrodose at 24 h (FIG. 11C) and 48 h (FIG. 11D) post-injection (FIGS. 11C and 11D Pearson product—moment correlation, the dashed line corresponds to the line of identity).

FIGS. 12A, 12B, and 12C are graphs showing time—activity curves obtained from positron emission tomography (PET) images from 5 min to 48 h post-injection in heart (FIG. 12A), kidney (FIG. 12B), and liver (FIG. 12C) from mice (n=3) that were administered a microdose of ⁶⁴Cu-MN-anti-miR10b. Error bars represent the standard deviation.

FIG. 13A is a graph showing the biodistribution of ⁶⁴Cu-MN-anti-miR10b injected at a microdose and a standard therapeutic dose (macrodose) measured at 24 h after injection. FIG. 13B is a graph showing the biodistribution of ⁶⁴Cu-MN-anti-miR10b injected at a microdose and a standard therapeutic dose (macrodose) measured at 48 h after injection. #Denotes organs with metastasis as detected by BLI. Results are expressed as % ID/g. Error bars represent the standard deviation. (t-test, *P<0.05, **P<0.01).

FIG. 14A is a set of in vivo PET-MRI maximum intensity projection (MIP) images of mice bearing metastatic breast adenocarcinoma 24 h after injection of a microdose or a macrodose of ⁶⁴Cu-MN-anti-miR10b. The yellow arrows point to bone or lymph node (LN) metastasis. FIG. 14B is a graph showing quantitation of ⁶⁴Cu-MN-anti-miR10b accumulation in metastatic (Mets+) and non-metastatic (Mets−) organs obtained from in vivo PET images at 24 h post-injection (% ID/cc). The high signal intensity in the metastatic organs compared to the non-metastatic organs confirms uptake of the therapeutic by the metastases. Open circles represent mice injected with a microdose and closed circles represent mice injected with a macrodose. FIG. 14C is a set of ex vivo PET-MRI images of bone and lymph node metastases. From left: in vivo BLI, ex vivo PET, and ex vivo white-light photograph of metastatic lesions. FIG. 14D is a set of ex vivo images of the biodistribution of ⁶⁴Cu-MN-anti-miR10b as visualized by PET 48 h after microdose injection and 24 h after macrodose injection.

FIG. 15 is a graph showing time-activity curves obtained from PET imaging from 5 min to 48 h post-injection of a microdose of 64 Cu-MN-anti-miR10b in metastatic (Mets+) and non-metastatic bones (Mets−) (n=3).

### DETAILED DESCRIPTION

The therapeutic nanoparticles described herein were discovered to detect cancer metastasis in a mammal and to decrease cancer cell invasion and cancer metastasis in a mammal. Therapeutic nanoparticles having these activities are provided herein as well as methods of decreasing cancer cell invasion or metastasis in a subject, methods of treating a metastatic cancer in a lymph node in a subject, and methods of detecting, diagnosing, and/or monitoring a metastatic cancer tissue in a subject by administering these therapeutic nanoparticles. Methods of preparing the therapeutic nanoparticles of the disclosure are also provided herein.

### Compositions

Provided herein are therapeutic nanoparticles that include a chelator that is covalently linked to the therapeutic nanoparticle and to a radiolabel and a nucleic acid molecule that is covalently linked to the therapeutic nanoparticle.

### Chelators

The therapeutic nanoparticles described herein can include at least one chelator covalently-linked to the therapeutic nanoparticle. In some embodiments, the chelator forms a stable complex with a radiolabel. In some embodiments, the chelator binds to a radiolabel.

In some embodiments, the therapeutic nanoparticles can include about 1 chelator to about 15 chelators (e.g., about 1 chelator to about 11 chelators, about 1 chelator to about 12 chelators, about 1 chelator to about 13 chelators, about 1 chelator to about 14 chelators, about 1 chelator to about 15 chelators, about 11 chelators to about 12 chelators, about 11 chelators to about 13 chelators, about 11 chelators to about 14 chelators, or about 11 chelators to about 15 chelators) covalently-linked to each therapeutic nanoparticle. In some embodiments, the therapeutic nanoparticles can include about 13 chelators covalently-linked to each therapeutic nanoparticle.

A variety of different chelators that can be covalently linked to the therapeutic nanoparticles are known in the art. Non-limiting examples of such chelators include 1,4,7-triazacyclononane,1-glutaric acid-4,7-acetic acid (NODAGA), 1,4,7,10-tetraazacyclododecane-1,4,7,10-tetrayl)tetraacetic acid (DOTA), 10-(2,6-dioxotetrahydro-2H-pyran-3-yl)-1,4,7,10-tetraazacyclododecane-1,4,7-triyl)triacetic acid (DOTA-GA), 2-[4,7,10-tris(carboxymethyl)-6-[(4-isothiocyanatophenyl)methyl]-1,4,7,10-tetrazacyclododec-1-yl]acetic acid (p-SCN-Bn)-DOTA, 2,2′-(1,4,8,11-tetraazabicyclo[6.6.2]hexadecane-4,11-diyl)diacetic acid (CB-TE2A), CB-TE1A1P, 1,4-bis(carboxymethyl)-6-[bis(carboxymethyl)]amino-6-methylperhydro-1,4-diazepine (AAZTA), 5-(8-methyl-3,6,10,13,16,19-hexaaza-bicyclo[6.6.6]icosan-1-ylamino)-5-oxopentanoic acid (MeCOSar), 2-S-(4-isothiocyanatobenzyl)-1,4,7-triazacyclononane-1,4,7-triacetic acid (p-SCN-Bn)-NOTA, 1,4,7-triazacyclononane-1,4,7-triacetic acid (NOTA), N,N′-bis-[2-hydroxy-5-(carboxyethyl)benzyl]ethylenediamine-N,N′-diacetic acid) (HBED-CC), tris(hydroxypyridinone) (THP), MASS, and desferoxamine (DFO).

Additional chelators are also described in Price and Ovig (Chem. Soc. Rev., 2014, 43, 260-290), Boros and Packard (Chem. Rev. 2019, 119, 2, 870-901), Barndt et al. (J. Nucl. Med., 2018, 59, 1500-1506), and Sneddon and Cornelissen (Curr. Opin. Chem. Biol., 2021, 63, 152-162) (each of which is incorporated by reference in its entirety).

In some embodiments, the chelator is attached to the therapeutic nanoparticle through a chemical moiety that contains a primary amine, secondary amine, an amide, a thioester, or a disulfide bond. Additional chemical moieties that can be used to covalently link a chelator to a therapeutic nanoparticle are known in the art.

A variety of different methods can be used to covalently link a chelator to a therapeutic nanoparticle. In some embodiments, the fluorophore is attached to the therapeutic nanoparticle through reaction of: an amine group (present in the chelator or on the therapeutic nanoparticle) with an active ester, carboxylate, isothiocyanate, or hydrazine (e.g., present in the chelator or on the therapeutic nanoparticle); through reaction of a carboxyl group (e.g., present in the chelator or on the therapeutic nanoparticle) in the presence of a carbodiimide; through reaction of a thiol (e.g., present in the chelator or on the therapeutic nanoparticle) in the presence of maleimide; through the reaction of a thiol (e.g., present in the chelator or on the therapeutic nanoparticle) in the presence of maleimide or acetyl bromide; or through the reaction of an azide (e.g., present in the chelator or on the therapeutic nanoparticle) in the presence of glutaraldehyde. Additional methods for attaching a chelator to a therapeutic nanoparticle are known in the art.

In some embodiments, the therapeutic nanoparticle does not include a chelator. In some embodiments, the therapeutic nanoparticle can include a radiolabel that is associated (e.g., covalently-linked) directly with the therapeutic nanoparticle.

### Radiolabels

The therapeutic nanoparticles described herein include a radiolabel. In some embodiments, the radiolabel can be associated with the nanoparticle. For example, in some embodiments, the radiolabel can be covalently or non-covalently bonded to the therapeutic nanoparticle via a linker. In some embodiments, the radiolabel can be covalently or non-covalently bonded directly to the therapeutic nanoparticle. In some embodiments, the radiolabel can be associated with the therapeutic nanoparticle or a composition or moiety surrounding the therapeutic nanoparticle (e.g., via van der Waals forces). In some embodiments, the radiolabel can be associated with the therapeutic nanoparticle without a chelator (e.g., wherein the therapeutic nanoparticle is chelator-free). In some embodiments, the radiolabel can be associated with the therapeutic nanoparticle with a chelator. In some embodiments, the radiolabel forms a stable complex with a chelator.

In some embodiments, the therapeutic nanoparticles can include about 1 radiolabel atom to about 15 radiolabel atoms (e.g., about 1 radiolabel atom to about 11 radiolabel atoms, about 1 radiolabel atom to about 12 radiolabel atoms, about 1 radiolabel atom to about 13 radiolabel atoms, about 1 radiolabel atom to about 14 radiolabel atoms, about 1 radiolabel atom to about 15 radiolabel atoms, about 13 radiolabel atoms to about 14 radiolabel atoms, about 13 radiolabel atoms to about 15 radiolabel atoms) per therapeutic nanoparticle. In some embodiments, the therapeutic nanoparticles can include about 14 radiolabel atoms associated (e.g., via a chelator) with each nanoparticle.

In some embodiments, the radiolabel has an emission energy (e.g., β⁺ energy) ranging from about 550 kiloelectron volts (keV) to about 3500 keV (e.g., about 550 keV to about 580 keV, about 550 keV to about 640 keV, about 550 keV to about 660 keV, about 550 keV to about 770 keV, about 550 keV to about 910 keV, about 579 keV to about 1200 keV, about 579 keV to about 1900 keV, about 579 keV to about 3500 keV). In some embodiments, the radiolabel has an emission energy of about 656 keV. In some embodiments, the radiolabel has an emission energy comparable (e.g., ±25 keV) to the emission energy of fluorine-18 (F-18). In some embodiments, the radiolabel has an emission energy of about 656 keV.

In some embodiments, the radiolabel has a half-life that enables an adequate assessment of the slow pharmacokinetics of macromolecules and/or blood-pool agents. In some embodiments, the radiolabel has a half-life ranging from about 10 minutes to about 80 hours (e.g., about 10 minutes to about 13 hours, about 70 minutes to about 13 hours, about 110 minutes to about 13 hours, about 13 hours to about 26 hours, about 13 hours to about 80 hours). In some embodiments, the radiolabel has a half-life of about 12.7 hours.

A variety of different radiolabels that can be associated (e.g., covalently-linked or non-covalently-linked) to the therapeutic nanoparticles are known in the art. Non-limiting examples of such chelators include copper-64 (Cu-64), F-18, scandium-44 (Sc-44), cobalt-55 (Co-55), niobium-90 (Nb-90), rhenium-188 (Re-188), thallium-201 (T1-201), copper-67 (Cu-67), yttrium-90 (Y-90), terbium-161 (Tb-161), lutetium-177 (Lu-177), bismuth-213 (Bi-213), lead-212 (Pb-212), actinium-225 (Ac-225), and zirconium-89 (Zr). In some embodiments, the radiolabel is Cu-64.

### Nucleic Acids

The therapeutic nanoparticles provided herein contain at least one nucleic acid molecule covalently-linked to the nanoparticle that contains at least 10 (e.g., at least 11, 12, 13, 14, 15, 16, 17, 18, 19, 20, 21, or 22) contiguous nucleotides within the sequence of precursor microRNA-10b (miR-10b). In some embodiments, the nucleic acid molecule contains the sequence of precursor miR-10b. In some embodiments, the therapeutic nanoparticles can contain a sequence that is complementary to at least 10 (e.g., at least 11,12,13,14,15,16,17,18,19,20,21,22, or 23) contiguous nucleotides within the sequence of precursor miR-10b.

In some embodiments, the nucleic acid molecule in the therapeutic nanoparticles contains the sequence or sequences of miR-21, miR-15, miR-16, miR-17, miR-18, miR-19a, miR-19b, miR-20, miR-92, miR-106, miR-191, miR-125b, miR-155, miR-569, miR-196b, or any combinations thereof. In some embodiments, the nucleic acid contained in the therapeutic nanoparticles can contain a sequence of at least 10 (e.g., at least 11, 12, 13, 14, 15, 16, 17, 18, 19, 20, 21, 22, or 23) contiguous nucleotides within a sequence complementary to mature human miR-21, miR-15, miR-16, miR-17, miR-18, miR-19a, miR-19b, miR-20, miR-92, miR-106, miR-191, miR-125b, miR-155, miR-569, miR-196b, or any combinations thereof. In some embodiments, the therapeutic nanoparticles can contain a sequence that is complementary to at least 10 (e.g., at least 11,12,13,14,15,16,17,18,19,20,21,22, or 23) contiguous nucleotides within the sequence of mature human miR-21, miR-15, miR-16, miR-17, miR-18, miR-19a, miR-19b, miR-miR-92, miR-106, miR-191, miR-125b, miR-155, miR-569, miR-196b, or any combinations thereof.

The attached nucleic acid can be single-stranded or double-stranded. In some embodiments, the nucleic acid contains a portion of the sequence of precursor miR-10b and has a total length of between 23 nucleotides and 50 nucleotides (e.g., between 23-30 nucleotides, between 30-40 nucleotides, and between 40-50 nucleotides). In some embodiments, the nucleic acid contains the sequence that is complementary to at least a portion of the sequence of precursor miR-10b and has a total length of between 22 nucleotides and 50 nucleotides (e.g., between 22-30 nucleotides, between 30-40 nucleotides, and between 40-50 nucleotides). In some embodiments, the nucleic acid can be an antisense RNA (e.g., an antagomir).

Antisense nucleic acid molecules can be covalently linked to the therapeutic nanoparticles described herein. For example, in some embodiments, the nucleic acid molecules can include at least a portion of the sequence (e.g., at least 10 nucleotides) that is complementary to the sequence of mature human miR-10b. in some embodiments, the nucleic acid molecules can include at least a portion of the sequence (e.g., at least 10 nucleotides) that is complementary to the sequence of the minor form of mature human miR-10b. In some embodiments, the therapeutic nanoparticles can include a nucleic acid molecule that targets human miR-10b for down-regulation.

In some embodiments, the therapeutic nanoparticles can include any of a number of appropriate antisense molecules (e.g., antisense molecules to target mature human miR-10b). For example, an antisense nucleic acid that targets miR-10b can contain a sequence complementary to at least 10 (e.g., at least 15 or 20) contiguous nucleotides present in a sequence for miR-10b known in the art.

An antisense nucleic acid can be constructed using chemical synthesis and enzymatic ligation reactions using procedures known in the art. For example, an antisense nucleic acid (e.g., an antisense oligonucleotide) can be chemically synthesized using naturally occurring nucleotides or modified nucleotides (e.g., any of the modified oligonucleotides described herein) designed to increase the biological stability of the molecules or to increase the physical stability of the duplex formed between the antisense and sense nucleic acids, e.g., phosphorothioate derivatives and acridine-substituted nucleotides can be used. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest). In some embodiments, the antisense nucleic acid molecules described herein can hybridize to a target nucleic acid by conventional nucleotide complementarities and form a stable duplex.

An antisense nucleic acid molecule can be an α-anomeric nucleic acid molecule. An α-anomeric nucleic acid molecule forms specific double-stranded hybrids with complementary RNA in which, contrary to the usual β-units, the strands run parallel to each other (Gaultier et al., Nucleic Acids Res. 15:6625-6641, 1987). The antisense nucleic acid molecule can also comprise a 2′-O-methylribonucleotide (Inoue et al., Nucleic Acids Res., 15:6131-6148, 1987) or a chimeric RNA-DNA analog (Inoue et al., FEBS Lett. 215:327-330, 1987).

In some embodiments, the nucleic acid molecule can contain at least one modified nucleotide (a nucleotide containing a modified base or sugar). In some embodiments, the nucleic acid molecule can contain at least one modification in the phosphate (phosphodiester) backbone. The introduction of these modifications can increase the stability, or improve the hybridization or solubility of the nucleic acid molecule.

The molecules described herein can contain one or more (e.g., two, three, four, of five) modified nucleotides. The modified nucleotides can contain a modified base or a modified sugar. Non-limiting examples of modified bases include: 8-oxo-N ⁶-methyladenine, 7-deazaxanthine, 7-deazaguanine, N ⁴, N ⁴-ethanocytosin, N ⁶, N ⁶-ethano-2,6-diaminopurine, 5-(C ³-C ⁶)-alkynyl-cytosine, pseudoisocytosine, 2-hydroxy-5-methyl-4-triazolopyridin, isocytosine, isoguanine, 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine.

Additional non-limiting examples of modified bases include those nucleobases described in U.S. Pat. Nos. 5,432,272 and 3,687,808 (herein incorporated by reference), Freier et al., Nucleic Acid Res. 25:4429-4443, 1997; Sanghvi, Antisense Research and Application, Chapter 15, Ed. S. T. Crooke and B. Lebleu, CRC Press, 1993; Englisch, et al., Angewandte Chemie 30:613-722, 1991, Kroschwitz, Concise Encyclopedia of Polymer Science and Engineering, John Wiley & Sons, pp. 858-859, 1990; and Cook, Anti-Cancer Drug Design 6:585-607, 1991. Additional non-limiting examples of modified bases include universal bases (e.g., 3-nitropyrole and 5-nitroindole). Other modified bases include pyrene and pyridyloxazole derivatives, pyrenyl, pyrenylmethylglycerol derivatives, and the like. Other preferred universal bases include pyrrole, diazole, or triazole derivatives, including those universal bases known in the art.

In some embodiments, the modified nucleotide can contain a modification in its sugar moiety. Non-limiting examples of modified nucleotides that contain a modified sugar are locked nucleotides (LNAs). LNA monomers are described in WO 99/14226 and U.S. Patent Application Publications Nos. 20110076675, 20100286044, 20100279895, 20100267018, 20100261175, 20100035968, 20090286753, 20090023594, 20080096191, 20030092905, 20020128381, and 20020115080 (herein incorporated by reference). Additional non-limiting examples of LNAs are disclosed in U.S. Pat. Nos. 6,043,060, 6,268,490, WO 01/07455, WO 01/00641, WO 98/39352, WO WO 00/56748, and WO 00/66604 (herein incorporated by reference), as well as in Morita et al., Bioorg. Med. Chem. Lett. 12(1):73-76, 2002; Hakansson et al., Bioorg. Med. Chem. Lett. 11(7):935-938, 2001; Koshkin et al., J. Org. Chem. 66(25):8504-8512, 2001; Kvaerno et al., J. Org. Chem. 66(16):5498-5503, 2001; Hakansson et al., J. Org. Chem. 65(17):5161-5166, 2000; Kvaerno et al., J. Org. Chem. 65(17):5167-5176, 2000; Pfundheller et al., Nucleosides Nucleotides 18(9):2017-2030, 1999; and Kumar et al., Bioorg. Med. Chem. Lett. 8(16):2219-2222, 1998. In some embodiments, the modified nucleotide is an oxy-LNA monomer, such as those described in WO 03/020739.

Modified nucleotides can also include antagomirs (2′-O-methyl-modified, cholesterol-conjugated single stranded RNA analogs); ALN (α-L-LNA); ADA (2′-N-adamantylmethylcarbonyl-2′-amino-LNA); PYR (2′-N-pyrenyl-1-methyl-2′-amino-LNA); OX (oxetane-LNA); ENA (2′-O, 4″-C-ethylene bridged nucleic acid); AENA (2′-deoxy-2′-N, 4′-C-ethylene-LNA); CLNA (2′,4′-carbocyclic-LNA); and CENA (2′,4′-carbocyclic-ENA); HM-modified DNAs (4′-C-hydroxymethyl-DNA); 2′-substituted RNAs (with 2′-O-methyl, 2′-fluoro, 2′-aminoethoxymethyl, 2′-aminopropoxymethyl, 2′-aminoethyl, 2′-guanidinoethyl, 2′-cyanoethyl, 2′-aminopropyl); and RNAs with radical modifications of the ribose sugar ring, such as Unlocked Nucleic Acid (UNA), Altritol Nucleic Acid (ANA) and Hexitol Nucleic Acid (HNA) (see, Bramsen et al., Nucleic Acids Res. 37:2867-81, 2009).

The molecules described herein can also contain a modification in the phosphodiester backbone. For example, at least one linkage between any two contiguous (adjoining) nucleotides in the molecule can be connected by a moiety containing 2 to 4 groups/atoms selected from the group of: —CH₂—, —O—, —S—, —NR ^{H},—>C═O, >C═NR ^{H}, >C═S, —Si(R″)₂—, —SO—, —S(O)₂—, —P(O)₂—, —PO(BH₃)—, —P(O,S)—, —P(S)₂—, —PO(R″)—, —PO(OCH₃)—, and —PO(NHR^{H})—, where R ^{H} is selected from hydrogen and C₁₋₄-alkyl, and R″ is selected from C₁₋₆-alkyl and phenyl. Illustrative examples of such linkages are —CH₂—CH₂—CH₂—, —CH₂—CO—CH₂—, —CH₂—CHOH—CH₂—, —O—CH₂—O—, —O—CH₂—CH₂—, —O—CH₂—CH═(including R ⁵ when used as a linkage to a succeeding monomer), —CH₂—CH₂—O—, NR^{H}—CH₂—CH₂—, —CH₂—CH₂—NR ^{H}—, —CH₂—NR ^{H}—CH₂—, —O—CH₂—CH₂—NR ^{H}—, —NR ^{H}—COO—, —NR ^{H}—CO—NR ^{H}—, —NR ^{H}—CS—NR ^{H}—, —NR^{H}—C(═NR^{H}—NR^{H}—, —NR^{H}—CO—CH₂—NR ^{H}—, —O—CO—O—, —O—CO—CH₂—O—, —O—CH₂—CO—O—, —CH₂—CO—NR^{H}—, —O—CO—NR^{H}—, —NR^{H}—CO—CH₂—, —O—CH₂—CO—NR^{H}—, —O—CH₂—CH₂—NR^{H}—, CH═N—O—, —CH₂—NR^{H}—O—, —CH₂—O—N═(including R ⁵ when used as a linkage to a succeeding monomer), —CH₂—O—NR ^{H}—, —CO—NR ^{H}—CH₂—, —CH₂—NR ^{H}—O—, —CH₂—NR ^{H}—CO—, —O—NR ^{H}—CH₂—, —O—NR ^{H}, —O—CH₂—S—, —S—CH₂—O—, —CH₂—CH₂—S—, —O—CH₂—CH₂—S—, —S—CH₂—CH═(including R ⁵ when used as a linkage to a succeeding monomer), —S-CH₂—CH₂—, —S—CH₂—CH₂—O—, —S—CH₂—CH₂—S—, —CH₂—S—CH₂—, CH₂—SO—CH₂—, —CH₂—SO₂—CH₂—, —O—SO—O—, —O—S(O)₂—O—, —O—S(O)₂—CH₂—, —O—S(O)₂—NR ^{H}—, —NR^{H}—S(O)₂—CH₂—, —O—S(O)₂—CH₂—, —O—P(O)₂—O—, —O—P(O,S)—O—, —O—P(S)₂—O—, —S—P(O)₂—O—, —S—P(O,S)—O—, —S—P(S)₂—O—, —O—P(O,S)—S—, —O—P(S)₂—S—, —S—P(O)₂—S—, —S—P(O,S)—S—, —S—P(S)₂—S—, —O—PO(R″)—O—, —O—PO(OCH₃)—O—, —O—PO—(OCH₂CH₃)—O—, —O—PO(OCH₂S—R)—O—, —O—PO(BH₃)—O—, —O—PO(NHR^{N})—O—, —O—P(O)₂—NR^{H}—, —NR^{H}—P(O)₂—O—, —O—P(O,NR^{H})₂—O—, —CH₂—P(O)₂—O—, —O—P(O)₂—CH₂—, and —O—Si(R″)₂—O—; among which —CH₂—CO—NR^{H}—, —CH₂—NR ^{H}—O—, —S—CH₂—O—, —O—P(O)₂—O—, —O—P(O,S)—O—, —O—P(S)₂—O—, —NR ^{H}—P(O)₂—O—, —O—P(O,NR^{H})—O—, —O—PO(R″)—O—, —O—PO(CH₃)—O—, and —O—PO(NHR^{N})—O—, where R H is selected form hydrogen and C₁₋₄-alkyl, and R″ is selected from C₁₋₆-alkyl and phenyl. Further illustrative examples are given in Mesmaeker et. al., Curr. Opin. Struct. Biol. 5:343-355, 1995; and Freier et al., Nucleic Acids Research 1997. The left-hand side of the inter-nucleoside linkage is bound to the 5-membered ring as substituent P* at the 3′-position, whereas the right-hand side is bound to the 5′-position of a preceding monomer.

In some embodiments, the deoxyribose phosphate backbone of the nucleic acid can be modified to generate peptide nucleic acids (see Hyrup et al., Bioorganic & Medicinal Chem. 4(1): 5-23, 1996). Peptide nucleic acids (PNAs) are nucleic acid mimics, e.g., DNA mimics, in which the deoxyribose phosphate backbone is replaced by a pseudopeptide backbone and only the four natural nucleobases are retained. The neutral backbone of PNAs allows for specific hybridization to DNA and RNA under conditions of low ionic strength. The synthesis of PNA oligomers can be performed using standard solid phase peptide synthesis protocols, e.g., as described in Hyrup et al., 1996, supra; Perry-O'Keefe et al., Proc. Natl. Acad. Sci. U.S.A. 93:14670-675, 1996.

PNAs can be modified, e.g., to enhance their stability or cellular uptake, by attaching lipophilic or other helper groups to PNA, by the formation of PNA-DNA chimeras, or by the use of liposomes or other techniques of delivery known in the art. For example, PNA-DNA chimeras can be generated which may combine the advantageous properties of PNA and DNA. Such chimeras allow DNA recognition enzymes, e.g., RNAse H, to interact with the DNA portion while the PNA portion would provide high binding affinity and specificity. PNA-DNA chimeras can be linked using linkers of appropriate lengths selected in terms of base stacking, number of bonds between the nucleobases, and orientation (Hyrup, 1996, supra). The synthesis of PNA-DNA chimeras can be performed as described in Hyrup, 1996, supra, and Finn et al., Nucleic Acids Res. 24:3357-63, 1996. For example, a DNA chain can be synthesized on a solid support using standard phosphoramidite coupling chemistry and modified nucleoside analogs. Compounds such as 5′-(4-methoxytrityl)amino-5′-deoxy-thymidine phosphoramidite can be used as a link between the PNA and the 5′ end of DNA (Mag et al., Nucleic Acids Res., 17:5973-88, 1989). PNA monomers are then coupled in a stepwise manner to produce a chimeric molecule with a 5′ PNA segment and a 3′ DNA segment (Finn et al., Nucleic Acids Res. 24:3357-63, 1996). Alternatively, chimeric molecules can be synthesized with a 5′ DNA segment and a 3′ PNA segment (Peterser et al., Bioorganic Med. Chem. Lett. 5:1119-11124, 1975).

In some embodiments, any of the nucleic acids described herein can be modified at either the 3′ or 5′ end (depending on how the nucleic acid is covalently-linked to the therapeutic nanoparticle) by any type of modification known in the art. For example, either end may be capped with a protecting group, attached to a flexible linking group, or attached to a reactive group to aid in attachment to the substrate surface (the polymer coating). Non-limiting examples of 3′ or 5′ blocking groups include: 2-amino-2-oxyethyl, 2-aminobenzoyl, 4-aminobenzoyl, acetyl, acetyloxy, (acetylamino)methyl, 3-(9-acridinyl), tricyclo[3.3.1.1(3,7)]dec-1-yloxy, 2-aminoethyl, propenyl, (9-anthracenylmethoxy)carbonyl, (1,1-dimethylpropoxy)carbonyl, (1,1-dimethylpropoxy)carbonyl, [1-methyl-1-[4-(phenylazo)phenyl] ethoxy] carbonyl, bromoacetyl, (benzoylamino)methyl, (2-bromoethoxy)carbonyl, (diphenylmethoxy)carbonyl, 1-methyl-3-oxo-3-phenyl-1-propenyl, (3-bromo-2-nitrophenyl)thio, (1,1-dimethylethoxy)carbonyl, [[(1,1-dimethylethoxy)carbonyl] amino]ethyl, 2-(phenylmethoxy)phenoxy, (1=[1,1′-biphenyl]-4-yl-1-methylethoxy) carbonyl, bromo, (4-bromophenyl)sulfonyl, 1H-benzotriazol-1-yl, [(phenylmethyl) thio]carbonyl, [(phenylmethyl)thio]methyl, 2-methylpropyl, 1,1-dimethylethyl, benzoyl, diphenylmethyl, phenylmethyl, carboxyacetyl, aminocarbonyl, chlorodifluoroacetyl, trifluoromethyl, cyclohexylcarbonyl, cycloheptyl, cyclohexyl, cyclohexylacetyl, chloro, carboxymethyl, cyclopentylcarbonyl, cyclopentyl, cyclopropylmethyl, ethoxycarbonyl, ethyl, fluoro, formyl, 1-oxohexyl, iodo, methyl, 2-methoxy-2-oxoethyl, nitro, azido, phenyl, 2-carboxybenzoyl, 4-pyridinylmethyl, 2-piperidinyl, propyl, 1-methylethyl, sulfo, and ethenyl. Additional examples of 5′ and 3′ blocking groups are known in the art. In some embodiments, the 5′ or 3′ blocking groups prevent nuclease degradation of the molecule.

In some embodiments, the nucleic acid molecule in the therapeutic nanoparticles can be a small interfering RNA (siRNA). RNAi is a process in which RNA is degraded in host cells. To decrease expression of an RNA, double-stranded RNA (dsRNA) containing a sequence corresponding to a portion of the target RNA (e.g., mature human miR-10b) is introduced into a cell. The dsRNA is digested into 21-23 nucleotide-long duplexes called short interfering RNAs (or siRNAs), which bind to a nuclease complex to form what is known as the RNA-induced silencing complex (or RISC). The RISC targets the endogenous target RNA by base pairing interactions between one of the siRNA strands and the endogenous RNA. It then cleaves the endogenous RNA about 12 nucleotides from the 3′ terminus of the siRNA (see Sharp et al., Genes Dev. 15:485-490, 2001, and Hammond et al., Nature Rev. Gen. 2:110-119, 2001).

Standard molecular biology techniques can be used to generate siRNAs. Short interfering RNAs can be chemically synthesized, recombinantly produced, e.g., by expressing RNA from a template DNA, such as a plasmid, or obtained from commercial vendors such as Dharmacon. The RNA used to mediate RNAi can include modified nucleotides (e.g., any of the modified nucleotides described herein), such as phosphorothioate nucleotides. The siRNA molecules used to decrease the levels of mature human miR-10b can vary in a number of ways. For example, they can include a 3′ hydroxyl group and strands of 21, 22, or 23 consecutive nucleotides. They can be blunt ended or include an overhanging end at either the 3′ end, the 5′ end, or both ends. For example, at least one strand of the RNA molecule can have a 3′ overhang from about 1 to about 6 nucleotides (e.g., 1-5, 1-3, 2-4 or 3-5 nucleotides (whether pyrimidine or purine nucleotides) in length. Where both strands include an overhang, the length of the overhangs may be the same or different for each strand.

To further enhance the stability of the RNA duplexes, the 3′ overhangs can be stabilized against degradation (by, e.g., including purine nucleotides, such as adenosine or guanosine nucleotides, or replacing pyrimidine nucleotides with modified nucleotides (e.g., substitution of uridine two-nucleotide 3′ overhangs by 2′-deoxythymidine is tolerated and does not affect the efficiency of RNAi). Any siRNA can be used provided it has sufficient homology to the target of interest. There is no upper limit on the length of the siRNA that can be used (e.g., the siRNA can range from about 21-50, 50-100, 100-250, 250-500, or 500-1000 base pairs).

In some embodiments, the nucleic acid molecule in the therapeutic nanoparticles can be an miRNA mimic. Non-limiting examples of the miRNA mimic include miRNA mimics of let7, miR-34a, miR-200c, miR-221/222, miR-126, miR-29, or any combinations thereof. In some embodiments, the nucleic acid molecule in the therapeutic nanoparticles can be an immunostimulatory nucleic acid. In some embodiments, the immunostimulatory nucleic acid is a an immunostimulatory RNA, and immunostimulatory DNA, or a combination thereof.

The nucleic acids described herein can be synthesized using any methods known in the art for synthesizing nucleic acids (see, e.g., Usman et al., J. Am. Chem. Soc. 109:7845, 1987; Scaringe et al., Nucleic Acid Res. 18:5433, 1990; Wincott et al., Methods Mol. Biol. 74:59, 1997; and Milligan, Nucleic Acid Res. 21:8783, 1987). These typically make use of common nucleic acid protecting and coupling groups. Synthesis can be performed on commercial equipment designed for this purpose, e.g., a 394 Applied Biosystems, Inc. synthesizer, using protocols supplied by the manufacturer. Additional methods for synthesizing the molecules described herein are known in the art. Alternatively, the nucleic acids can be specially ordered from commercial vendors that synthesize oligonucleotides.

In some embodiments, the nucleic acid is attached to the therapeutic nanoparticle at its 5′ end. In some embodiments, the nucleic acid is attached to the therapeutic nanoparticle at its 3′ end. In some embodiments, the nucleic acid is attached to the therapeutic nanoparticle through a base present in the nucleic acid.

In some embodiments, the nucleic acid (e.g., any of the nucleic acids described herein) is attached to the therapeutic nanoparticle (e.g., to the polymer coating of the therapeutic nanoparticle) through a chemical moiety that contains a thioether bond or a disulfide bond. In some embodiments, the nucleic acid is attached to the therapeutic nanoparticle through a chemical moiety that contains an amide bond. Additional chemical moieties that can be used to covalently link a nucleic acid to a therapeutic nanoparticle are known in the art.

A variety of different methods can be used to covalently link a nucleic acid to a therapeutic nanoparticle. Non-limiting examples of methods that can be used to link a nucleic acid to a magnetic particle are described in EP 0937097; U.S. RE41005; Lund et al., Nucleic Acid Res. 16:10861, 1998; Todt et al., Methods Mol. Biol. 529:81-100, 2009; Brody et al., J. Biotechnol. 74:5-13, 2000; Ghosh et al., Nucleic Acids Res. 15:5353-5372, 1987; U.S. Pat. Nos. 5,900,481; 7,569,341; 6,995,248; 6,818,394; 6,811,980; 5,900,481; and 4,818,681 (each of which is incorporated by reference in its entirety). In some embodiments, carbodiimide is used for the end-attachment of a nucleic acid to a therapeutic nanoparticle. In some embodiments, the nucleic acid is attached to the therapeutic nanoparticle through the reaction of one of its bases with an activated moiety present on the surface of the therapeutic nanoparticle (e.g., the reaction of an electrophilic base with a nucleophilic moiety on the surface of the therapeutic nanoparticle, or the reaction of a nucleophilic base with an electrophilic residue on the surface of the therapeutic nanoparticle). In some embodiments, a 5′—NH₂ modified nucleic acid is attached to a therapeutic nanoparticle containing CNBr-activated hydroxyl groups (see, e.g., Lund et al., supra). Additional methods for attaching an amino-modified nucleic acid to a therapeutic nanoparticle are described below. In some embodiments, a ₅′-phosphate nucleic acid is attached to a therapeutic nanoparticle containing hydroxyl groups in the presence of a carbodiimide (see, e.g., Lund et al., supra). Other methods of attaching a nucleic acid to a therapeutic nanoparticle include carbodiimide-mediated attachment of a 5′-phosphate nucleic acid to a NH₂ group on a therapeutic nanoparticle, and carbodiimide-mediated attachment of a 5′-NH₂ nucleic acid to a therapeutic nanoparticle having carboxyl groups (see, e.g., Lund et al., supra).

In exemplary methods, a nucleic acid can be produced that contains a reactive amine or a reactive thiol group. The amine or thiol in the nucleic acid can be linked to another reactive group. The two common strategies to perform this reaction are to link the nucleic acid to a similar reactive moiety (amine to amine or thiol to thiol), which is called homobifunctional linkage, or to link to the nucleic acid to an opposite group (amine to thiol or thiol to amine), known as heterobifunctional linkage. Both techniques can be used to attach a nucleic acid to a therapeutic nanoparticle (see, for example, Misra et al., Bioorg. Med. Chem. Lett. 18:5217-5221, 2008; Mirsa et al., Anal. Biochem. 369:248-255, 2007; Mirsa et al., Bioorg. Med. Chem. Lett. 17:3749-3753, 2007; and Choithani et al., Methods Mol Biol. 381:133-163, 2007).

Traditional attachment techniques, especially for amine groups, have relied upon homobifunctional linkages. One of the most common techniques has been the use of bisaldehydes such as glutaraldehyde. Disuccinimydyl suberate (DSS), commercialized by Syngene (Frederick, MD) as synthetic nucleic acid probe (SNAP) technology, or the reagent p-phenylene diisothiocyanate can also be used to generate a covalent linkage between the nucleic acid and the therapeutic nanoparticle. N,N′-o-phenylenedimaleimide can be used to cross-link thiol groups. With all of the homobifunctional cross-linking agents, the nucleic acid is initially activated and then added to the therapeutic nanoparticle (see, for example, Swami et al., Int. J. Pharm. 374:125-138, 2009, Todt et al., Methods Mol. Biol. 529:81-100, 2009; and Limanski\check{i}, Biofizika 51:225-235, 2006).

Heterobifunctional linkers can also be used to attach a nucleic acid to a therapeutic nanoparticle. For example, N-succinidimidyl-3-(2-pyridyldithio)proprionate (SPDP) initially links to a primary amine to give a dithiol-modified compound. This can then react with a thiol to exchange the pyridylthiol with the incoming thiol (see, for example, Nostrum et al., J. Control Release 15;153(1):93-102, 2011, and Berthold et al., Bioconjug. Chem. 21:1933-1938, 2010).

An alternative approach for thiol use has been a thiol-exchange reaction. If a thiolated nucleic acid is introduced onto a disulfide therapeutic nanoparticle, a disulfide-exchange reaction can occur that leads to the nucleic acid being covalently bonded to the therapeutic nanoparticle by a disulfide bond. A multitude of potential cross-linking chemistries are available for the heterobifunctional cross-linking of amines and thiols. Generally, these procedures have been used with a thiolated nucleotide. Reagents typically employed have been NHS (N-hydroxysuccinimide ester), MBS (m-maleimidobenzoyl-N-succinimide ester), and SPDP (a pyridyldisulfide-based system). The heterobifunctional linkers commonly used rely upon an aminated nucleic acid. Additional methods for covalently linking a nucleic acid to a therapeutic nanoparticle are known in the art.

### Nanoparticles

In some embodiments, the therapeutic nanoparticles can have a diameter of between about 2 nm to about 200 nm (e.g., between about 10 nm to about 30 nm, between about 5 nm to about 25 nm, between about 10 nm to about 25 nm, between about 15 nm to about 25 nm, between about 20 nm and about 25 nm, between about 25 nm to about 50 nm, between about 50 nm and about 200 nm, between about 70 nm and about 200 nm, between about 80 nm and about 200 nm, between about 100 nm and about 200 nm, between about 140 nm to about 200 nm, and between about 150 nm to about 200 nm).

In some embodiments, the therapeutic nanoparticles can have a diameter that is about 18% to about 28% (e.g., about 18% to about 23%, about 20% to about 23%, about 23% to about 25%, about 23% to about 28%) greater than a diameter of a nanoparticle that does not include a radiolabel. In some embodiments, the therapeutic nanoparticles can have a diameter that is about 23% greater than a diameter of a nanoparticle that does not include a radiolabel. In some embodiments, the therapeutic nanoparticles can accumulate in a metastatic tissue of a subject at a similar rate than metastatic-targeting nanoparticles that do not include a radiolabel. In some embodiments, the therapeutic nanoparticles exhibit about the same accumulation in a metastatic tissue of a subject as compared to metastatic-targeting nanoparticles that do not include a radiolabel.

In some embodiments, the therapeutic nanoparticles provided herein can be spherical or ellipsoidal, or can have an amorphous shape. In some embodiments, the therapeutic nanoparticles provided herein can have a diameter (between any two points on the exterior surface of the therapeutic nanoparticle) of between about 2 nm to about 200 nm (e.g., between about 10 nm to about 200 nm, between about 2 nm to about 30 nm, between about 5 nm to about 25 nm, between about 10 nm to about 25 nm, between about 15 nm to about 25 nm, between about 20 nm to about 25 nm, between about 50 nm to about 200 nm, between about 70 nm to about 200 nm, between about 80 nm to about 200 nm, between about 100 nm to about 200 nm, between about 140 nm to about 200 nm, and between about 150 nm to about 200 nm). In some embodiments, therapeutic nanoparticles having a diameter of between about 2 nm to about 30 nm localize to the lymph nodes in a subject. In some embodiments, therapeutic nanoparticles having a diameter of between about 40 nm to about 200 nm localize to the liver.

In some embodiments, the therapeutic nanoparticles can be magnetic (e.g., include a core of a magnetic material). In some embodiments, any of the therapeutic nanoparticles described herein can contain a core of a magnetic material (e.g., a therapeutic magnetic nanoparticle). In some embodiments, the therapeutic nanoparticle can include an iron oxide core. In some embodiments, the magnetic material or particle can contain a diamagnetic, paramagnetic, superparamagnetic, or ferromagnetic material that is responsive to a magnetic field. Non-limiting examples of therapeutic magnetic nanoparticles contain a core of a magnetic material containing a metal oxide selected from the group of: magnetite; ferrites (e.g., ferrites of manganese, cobalt, and nickel); Fe(II) oxides, and hematite, and metal alloys thereof. The core of magnetic material can be formed by converting metal salts to metal oxides using methods known in the art (e.g., Kieslich et al., Inorg. Chem. 2011). In some embodiments, the nanoparticles contain cyclodextrin gold or quantum dots. Non-limiting examples of methods that can be used to generate therapeutic magnetic nanoparticles are described in Medarova et al., Methods Mol. Biol. 555:1-13, 2009; and Medarova et al., Nature Protocols 1:429-431, 2006. Additional magnetic materials and methods of making magnetic materials are known in the art. In some embodiments of the methods described herein, the position or localization of therapeutic magnetic nanoparticles can be imaged in a subject (e.g., imaged in a subject following the administration of one or more doses of a therapeutic magnetic nanoparticle).

In some embodiments, the therapeutic nanoparticles described herein do not contain a magnetic material. In some embodiments, a therapeutic nanoparticle can contain, in part, a core of containing a polymer (e.g., poly(lactic-co-glycolic acid)). Skilled practitioners may appreciate that any number of art known materials can be used to prepare nanoparticles, including, but are not limited to, gums (e.g., Acacia, Guar), chitosan, gelatin, sodium alginate, and albumin. Additional polymers that can be used to generate the therapeutic nanoparticles described herein are known in the art. For example, polymers that can be used to generate the therapeutic nanoparticles include, but are not limited to, cellulosics, poly(2-hydroxy ethyl methacrylate), poly(N-vinyl pyrrolidone), poly(methyl methacrylate), poly(vinyl alcohol), poly(acrylic acid), polyacrylamide, poly(ethylene-co-vinyl acetate), poly(ethylene glycol), poly(methacrylic acid), polylactides (PLA), polyglycolides (PGA), poly(lactide-co-glycolides) (PLGA), polyanhydrides, polyorthoesters, polycyanoacrylates, and polycaprolactones.

Skilled practitioners will appreciate that the material used in the composition of the nanoparticles, the methods for preparing, coating, and methods for controlling the size of the nanoparticles can vary substantially. However, these methods are well known to those in the art. Key issues include the biodegradability, toxicity profile, and pharmacokinetics/pharmacodynamics of the nanoparticles. The composition and/or size of the nanoparticles are key determinants of their biological fate. For example, larger nanoparticles are typically taken up and degraded by the liver, whereas smaller nanoparticles (<30 nm in diameter) typically circulate for a long time (sometimes over 24-hr blood half-life in humans) and accumulate in lymph nodes and the interstitium of organs with hyperpermeable vasculature, such as tumors.

### Polymer Coatings

The therapeutic nanoparticles described herein contain a polymer coating over the core magnetic material (e.g., over the surface of a magnetic material). The polymer material can be suitable for attaching or coupling one or more biological agents (e.g., such as any of the nucleic acids described herein). One of more biological agents (e.g., a nucleic acid, fluorophore, or targeting peptide) can be fixed to the polymer coating by chemical coupling (covalent bonds).

In some embodiments, the therapeutic nanoparticles are formed by a method that includes coating the core of magnetic material with a polymer that is relatively stable in water. In some embodiments, the therapeutic nanoparticles are formed by a method that includes coating a magnetic material with a polymer or absorbing the magnetic material into a thermoplastic polymer resin having reducing groups thereon. A coating can also be applied to a magnetic material using the methods described in U.S. Pat. Nos. 5,834,121, 5,356,713, 5,318,797, 5,283,079, 5,232,789, 5,091,206, 4,965,007, 4,774,265, 4,770,183, 4,654,267, 4,554,088, 4,490,436, 4,336,173, and 4,421,660; and WO (each disclosure of which is incorporated herein by reference).

Method for the synthesis of iron oxide nanoparticles include, for example, physical and chemical methods. For example, iron oxides can be prepared by co-precipitation of Fe ²⁺ and Fe ³⁺ salts in an aqueous solution. The resulting core consists of magnetite (Fe₃O₄), maghemite (γ-Fe₂O₃) or a mixture of the two. The anionic salt content (chlorides, nitrates, sulphates, etc.), the Fe ²⁺ and Fe ³⁺ ratio, pH and the ionic strength in the aqueous solution all play a role in controlling the size. It is important to prevent the oxidation of the synthesized nanoparticles and protect their magnetic properties by carrying out the reaction in an oxygen free environment under inert gas such as nitrogen or argon. The coating materials can be added during the co-precipitation process in order to prevent the agglomeration of the iron oxide nanoparticles into microparticles. Skilled practitioners will appreciate that any number of art known surface coating materials can be used for stabilizing iron oxide nanoparticles, among which are synthetic and natural polymers, such as, for example, polyethylene glycol (PEG), dextran, polyvinylpyrrolidone (PVP), fatty acids, polypeptides, chitosan, gelatin.

For example, U.S. Pat. No. 4,421,660 notes that polymer coated particles of an inorganic material are conventionally prepared by (1) treating the inorganic solid with acid, a combination of acid and base, alcohol or a polymer solution; (2) dispersing an addition polymerizable monomer in an aqueous dispersion of a treated inorganic solid and (3) subjecting the resulting dispersion to emulsion polymerization conditions. (col. 1, lines 21-27) U.S. Pat. No. 4,421,660 also discloses a method for coating an inorganic nanoparticles with a polymer, which comprises the steps of (1) emulsifying a hydrophobic, emulsion polymerizable monomer in an aqueous colloidal dispersion of discrete particles of an inorganic solid and (2) subjecting the resulting emulsion to emulsion polymerization conditions to form a stable, fluid aqueous colloidal dispersion of the inorganic solid particles dispersed in a matrix of a water-insoluble polymer of the hydrophobic monomer (col. 1, lines 42-50).

Alternatively, polymer-coated magnetic material can be obtained commercially that meets the starting requirements of size. For example, commercially available ultrasmall superparamagnetic iron oxide nanoparticles include NC100150 Injection (Nycomed Amersham, Amersham Health) and Ferumoxytol (AMAG Pharmaceuticals, Inc.).

Suitable polymers that can be used to coat the core of magnetic material include without limitation: polystyrenes, polyacrylamides, polyetherurethanes, polysulfones, fluorinated or chlorinated polymers such as polyvinyl chloride, polyethylenes, and polypropylenes, polycarbonates, and polyesters. Additional examples of polymers that can be used to coat the core of magnetic material include polyolefins, such as polybutadiene, polydichlorobutadiene, polyisoprene, polychloroprene, polyvinylidene halides, polyvinylidene carbonate, and polyfluorinated ethylenes. A number of copolymers, including styrene/butadiene, alpha-methyl styrene/dimethyl siloxane, or other polysiloxanes can also be used to coat the core of magnetic material (e.g., polydimethyl siloxane, polyphenylmethyl siloxane, and polytrifluoropropylmethyl siloxane). Additional polymers that can be used to coat the core of magnetic material include polyacrylonitriles or acrylonitrile-containing polymers, such as poly alpha-acrylanitrile copolymers, alkyd or terpenoid resins, and polyalkylene polysulfonates. In some embodiments, the polymer coating is dextran.

### Pharmaceutical Compositions

Also provided herein are pharmaceutical compositions that contain a therapeutic nanoparticle as described herein. Two or more (e.g., two, three, or four) of any of the types of therapeutic nanoparticles described herein can be present in a pharmaceutical composition in any combination. The pharmaceutical compositions can be formulated in any manner known in the art.

Pharmaceutical compositions are formulated to be compatible with their intended route of administration (e.g., intravenous, intraarterial, intramuscular, intradermal, subcutaneous, or intraperitoneal). In some embodiments, the compositions provided herein can include a pharmaceutically acceptable diluent (e.g., a sterile diluent). In some embodiments, the pharmaceutically acceptable diluent can be sterile water, sterile saline, a fixed oil, polyethylene glycol, glycerine, propylene glycol or other synthetic solvents, antibacterial or antifungal agents such as benzyl alcohol or methyl parabens, chlorobutanol, phenol, ascorbic acid, thimerosal, and the like, antioxidants such as ascorbic acid or sodium bisulfite, chelating agents such as ethylenediaminetetraacetic acid, buffers such as acetates, citrates, or phosphates, and isotonic agents such as sugars (e.g., dextrose), polyalcohols (e.g., mannitol or sorbitol), or salts (e.g., sodium chloride), or any combination thereof.

In some embodiments, the pharmaceutical compositions provided herein can include a pharmaceutically acceptable carrier. Liposomal suspensions can also be used as pharmaceutically acceptable carriers (see, e.g., U.S. Pat. No. 4,522,811). Preparations of the compositions can be formulated and enclosed in ampules, disposable syringes, or multiple dose vials. Where required (as in, for example, injectable formulations), proper fluidity can be maintained by, for example, the use of a coating such as lecithin, or a surfactant. Absorption of the therapeutic nanoparticles can be prolonged by including an agent that delays absorption (e.g., aluminum monostearate and gelatin). Alternatively, controlled release can be achieved by implants and microencapsulated delivery systems, which can include biodegradable, biocompatible polymers (e.g., ethylene vinyl acetate, polyanhydrides, polyglycolic acid, collagen, polyorthoesters, and polylactic acid; Alza Corporation and Nova Pharmaceutical, Inc.).

Compositions containing one or more of any of the therapeutic nanoparticles described herein can be formulated for parenteral (e.g., intravenous, intraarterial, intramuscular, intradermal, subcutaneous, or intraperitoneal) administration in dosage unit form (i.e., physically discrete units containing a predetermined quantity of active compound for ease of administration and uniformity of dosage). In some embodiments, the compositions containing one or more of any of the therapeutic nanoparticles described herein can be formulated into a dosage form that is an injectable, a tablet, a lyophilized powder, a suspension, or any combination thereof.

Toxicity and therapeutic efficacy of compositions can be determined by standard pharmaceutical procedures in cell cultures or experimental animals (e.g., monkeys). One can, for example, determine the LD50 (the dose lethal to 50% of the population) and the ED50 (the dose therapeutically effective in 50% of the population): the therapeutic index being the ratio of LD50:ED50. Agents that exhibit high therapeutic indices are preferred. Where an agent exhibits an undesirable side effect, care should be taken to minimize potential damage (i.e., reduce unwanted side effects). Toxicity and therapeutic efficacy can be determined by other standard pharmaceutical procedures.

Data obtained from cell culture assays and animal studies can be used in formulating an appropriate dosage of any given agent for use in a subject (e.g., a human). A therapeutically effective amount of the one or more (e.g., one, two, three, or four) therapeutic nanoparticles (e.g., any of the therapeutic nanoparticles described herein) will be an amount that treats decreases cancer cell invasion or metastasis in a subject having cancer (e.g., breast cancer) in a subject (e.g., a human), treats a metastatic cancer in a lymph node in a subject, decreases or stabilizes metastatic tumor size in a lymph node in a subject, decreases the rate of metastatic tumor growth in a lymph node in a subject, decreases the severity, frequency, and/or duration of one or more symptoms of a metastatic cancer in a lymph node in a subject in a subject (e.g., a human), or decreases the number of symptoms of a metastatic cancer in a lymph node in a subject (e.g., as compared to a control subject having the same disease but not receiving treatment or a different treatment, or the same subject prior to treatment).

The effectiveness and dosing of any of the therapeutic nanoparticles described herein can be determined by a health care professional using methods known in the art, as well as by the observation of one or more symptoms of a metastatic cancer in a lymph node in a subject (e.g., a human). Certain factors may influence the dosage and timing required to effectively treat a subject (e.g., the severity of the disease or disorder, previous treatments, the general health and/or age of the subject, and the presence of other diseases).

Exemplary doses include milligram or microgram amounts of any of the therapeutic nanoparticles described herein per kilogram of the subject's weight. For example, in some embodiments, the therapeutic nanoparticles can be administered to a subject at a dose that is less than about 0.020 mg/kg (e.g., about 0.001 mg/kg to about mg/kg, about 0.005 to about 0.010 mg/kg, about 0.010 mg/kg to about 0.015 mg/kg, about 0.011 mg/kg to about 0.015 mg/kg, about 0.012 mg/kg to about 0.015 mg/kg, about 0.013 mg/kg to about 0.015 mg/kg, about 0.012 mg/kg to about 0.016 mg/kg, about 0.013 mg/kg to about 0.017 mg/kg, about 0.013 mg/kg to about 0.018 mg/kg, or about 0.015 mg/kg to about 0.020 mg/kg). In some embodiments, the therapeutic nanoparticles can be administered to a subject at a dose that is less than about mg/kg. In some embodiments, the therapeutic nanoparticles can be administered to a subject at a dose that is less than about 0.014 mg/kg for imaging, detecting, diagnosing, and/or monitoring a metastatic cancer tissue in a subject.

In some embodiments, the therapeutic nanoparticles can be administered to a subject at a dose ranging from about 1 mg/kg to about 10 mg/kg (e.g., about 1 mg/kg to about 5 mg/kg, about 1 to about 7 mg/kg, about 2 mg/kg to about 7 mg/kg, about 2 mg/kg to about 8 mg/kg, about 2 mg/kg to about 9 mg/kg, about 2 mg/kg to about 10 mg/kg, about 3 mg/kg to about 5 mg/kg, about 3 mg/kg to about 6 mg/kg, about 3 mg/kg to about 7 mg/kg, about 3 mg/kg to about 8 mg/kg, about 3 mg/kg to about 9 mg/kg, about 3 mg/kg to about 10 mg/kg, about 4 mg/kg to about 5 mg/kg, about 4 mg/kg to about 6 mg/kg, about 4 mg/kg to about 7 mg/kg, about 4 mg/kg to about 8 mg/kg, about 4 mg/kg to about 9 mg/kg, about 4 mg/kg to about 10 mg/kg, about 5 mg/kg to about 6 mg/kg, about 5 mg/kg to about 7 mg/kg, about 5 mg/kg to about 8 mg/kg, about 5 mg/kg to about 9 mg/kg, about 5 mg/kg to about 10 mg/kg, about 6 mg/kg to about 7 mg/kg, about 6 mg/kg to about 8 mg/kg, about 6 mg/kg to about 9 mg/kg, about 6 mg/kg to about 10 mg/kg, about 7 mg/kg to about 8 mg/kg, about 7 mg/kg to about 9 mg/kg, or about 7 mg/kg to about 10 mg/kg). In some embodiments, the therapeutic nanoparticles can be administered to a subject at a dose of about 5 mg/kg to about 7 mg/kg. In some embodiments, the therapeutic nanoparticles can be administered to a subject at a dose that is less than about of about 5 mg/kg to about 7 mg/kg for treating a metastatic cancer and/or decreasing cell invasion or metastasis in a subject.

While these doses cover certain ranges, one of ordinary skill in the art will understand that therapeutic agents, including the therapeutic nanoparticles described herein, vary in their potency, and effective amounts can be determined by methods known in the art. Typically, relatively low doses are administered at first, and the attending health care professional (in the case of therapeutic application) or a researcher (when still working at the development stage) can subsequently and gradually increase the dose until an appropriate response is obtained. In addition, it is understood that the specific dose level for any particular subject will depend upon a variety of factors including the activity of the specific compound employed, the age, body weight, general health, gender, and diet of the subject, the time of administration, the route of administration, the rate of excretion, and the half-life of the therapeutic nanoparticles in vivo.

The pharmaceutical compositions can be included in a kit, container, pack, or dispenser together with instructions for administration.

### Methods of Treatment

The therapeutic nanoparticles described herein were discovered to decrease cancer cell invasion and to inhibit cancer cell metastasis. In view of this discovery, provided herein are methods of decreasing cancer cell invasion or metastasis in a subject, methods of treating a metastatic cancer in a lymph node in a subject, and methods of delivering a nucleic acid to a cell present in the lymph node of a subject. Specific embodiments and various aspects of these methods are described below.

### Methods of Treating Metastatic Cancer

Metastatic cancer is a cancer that originates from a cancer cell from a primary tumor that has migrated to a different tissue in the subject. In some embodiments, the cancer cell from the primary tumor can migrate to a different tissue in the subject by traveling through the blood stream or the lymphatic system of the subject. In some embodiments, the metastatic cancer is a metastatic cancer present in a lymph node in a subject.

The symptoms of metastatic cancer experienced by a subject depend on the site of metastatic tumor formation. Non-limiting symptoms of metastatic cancer in the brain of a subject include: headaches, dizziness, and blurred vision. Non-limiting symptoms of metastatic cancer in the liver of a subject include: weight loss, fever, nausea, loss of appetite, abdominal pain, fluid in the abdomen (ascites), jaundice, and swelling of the legs. Non-limiting symptoms of metastatic cancer in the bone of a subject include: pain and bone breakage following minor or no injury. Non-limiting symptoms of metastatic cancer in the lung of a subject include: non-productive cough, cough producing bloody sputum, chest pain, and shortness of breath.

A metastatic cancer can be diagnosed in a subject by a health care professional (e.g., a physician, a physician's assistant, a nurse, or a laboratory technician) using methods known in the art. For example, a metastatic cancer can be diagnosed in a subject, in part, by the observation or detection of at least one symptom of a metastatic cancer in a subject (e.g., any of those symptoms listed above). A metastatic cancer can also be diagnosed in a subject using a variety of imaging techniques (e.g., alone or in combination with the observance of one or more symptoms of a metastatic cancer in a subject). For example, the presence of a metastatic cancer (e.g., a metastatic cancer in a lymph node) can be detected in a subject using computer tomography, magnetic resonance imaging, positron emission tomography, and X-ray. A metastatic cancer (e.g., a metastatic cancer in a lymph node) can also be diagnosed by performing a biopsy of tissue from the subject (e.g., a biopsy of a lymph node from the subject).

A metastatic tumor can form in a variety of different tissues in a subject, including, but not limited to: brain, lung, liver, bone, peritoneum, adrenal gland, skin, and muscle. The primary tumor can be of any cancer type, including but not limited to: breast, colon, kidney, lung, skin, ovarian, pancreatic, rectal, stomach, thyroid, or uterine cancer.

Any one or more of the therapeutic nanoparticles described herein can be administered to a subject having a metastatic cancer. The one or more therapeutic nanoparticles can be administered to a subject in a health care facility (e.g., in a hospital or a clinic) or in an assisted care facility. In some embodiments, the subject has been previously diagnosed as having a cancer (e.g., a primary cancer). In some embodiments, the subject has been previously diagnosed as having a metastatic cancer (e.g., a metastatic cancer in the lymph node). In some embodiments, the subject has already received therapeutic treatment for the primary cancer. In some embodiments, the primary tumor of the subject has been surgically removed prior to treatment with one of the therapeutic nanoparticles described herein. In some embodiments, at least one lymph node has been removed from the subject prior to treatment with one of the therapeutic nanoparticles described herein. In some embodiments, the subject may be in a period of cancer remission.

In some embodiments, the administering of at least one therapeutic nanoparticle results in a decrease (e.g., a significant or observable decrease) in the size of a metastatic tumor present in a lymph node, a stabilization of the size (e.g., no significant or observable change in size) of a metastatic tumor present in a lymph node, or a decrease (e.g., a detectable or observable decrease) in the rate of the growth of a metastatic tumor present in a lymph node in a subject. A health care professional can monitor the size and/or changes in the size of a metastatic tumor present in a lymph node in a subject using a variety of different imaging techniques, including but not limited to: computer tomography, magnetic resonance imaging, positron emission tomography, and X-ray. For example, the size of a metastatic tumor present in a lymph node of a subject can be determined before and after therapy in order to determine whether there has been a decrease or stabilization in the size of the metastatic tumor in the subject in response to therapy. The rate of growth of a metastatic tumor in the lymph node of a subject can be compared to the rate of growth of a metastatic tumor in another subject or population of subjects not receiving treatment or receiving a different treatment. A decrease in the rate of growth of a metastatic tumor in the lymph node of a subject can also be determined by comparing the rate of growth of a metastatic tumor in a lymph node both prior to and following a therapeutic treatment (e.g., treatment with any of the therapeutic nanoparticles described herein). In some embodiments, the visualization of a metastatic tumor (e.g., a metastatic tumor in a lymph node) can be performed using imaging techniques that utilize a labeled probe or molecule that binds specifically to the cancer cells in the metastatic tumor (e.g., a labeled antibody that selectively binds to an epitope present on the surface of the primary cancer cell).

In some embodiments, the administering of at least one therapeutic nanoparticle to the subject results in a decrease in the risk of developing an additional metastatic tumor in a subject already having at least one metastatic tumor (e.g., a subject already having a metastatic tumor in a lymph node) (e.g., as compared to the rate of developing an additional metastatic tumor in a subject already having a similar metastatic tumor but not receiving treatment or receiving an alternative treatment). A decrease in the risk of developing an additional metastatic tumor in a subject already having at least one metastatic tumor can also be compared to the risk of developing an additional metastatic tumor in a population of subjects receiving no therapy or an alternative form of cancer therapy.

In some embodiments, administering a therapeutic nanoparticle to the subject decreases the risk of developing a metastatic cancer (e.g., a metastatic cancer in a lymph node) in a subject having (e.g., diagnosed as having) a primary cancer (e.g., a primary breast cancer) (e.g., as compared to the rate of developing a metastatic cancer in a subject having a similar primary cancer but not receiving treatment or receiving an alternative treatment). A decrease in the risk of developing a metastatic tumor in a subject having a primary cancer can also be compared to the rate of metastatic cancer formation in a population of subjects receiving no therapy or an alternative form of cancer therapy.

A health care professional can also assess the effectiveness of therapeutic treatment of a metastatic cancer (e.g., a metastatic cancer in a lymph node of a subject) by observing a decrease in the number of symptoms of metastatic cancer in the subject or by observing a decrease in the severity, frequency, and/or duration of one or more symptoms of a metastatic cancer in a subject. A variety of symptoms of a metastatic cancer are known in the art and are described herein. Non-limiting examples of symptoms of metastatic cancer in a lymph node include: pain in a lymph node, swelling in a lymph node, appetite loss, and weight loss.

In some embodiments, the administering can result in an increase (e.g., a significant increase) the chance of survival of a primary cancer or a metastatic cancer in a subject (e.g., as compared to a population of subjects having a similar primary cancer or a similar metastatic cancer but receiving a different therapeutic treatment or no therapeutic treatment). In some embodiments, the administering can result in an improved prognosis for a subject having a primary cancer or a metastatic cancer (e.g., as compared to a population of subjects having a similar primary cancer or a similar metastatic cancer but receiving a different therapeutic treatment or no therapeutic treatment).

### Methods of Decreasing Cancer Cell Invasion or Metastasis

Also provided are methods of decreasing (e.g., a significant or observable decrease) cancer cell invasion or metastasis in a subject that include administering at least one therapeutic nanoparticle described herein to the subject in an amount sufficient to decrease cancer cell invasion or metastasis in a subject.

In some embodiments of these methods, the cancer cell metastasis is from a primary tumor (e.g., any of the primary tumors described herein) to a secondary tissue (e.g., a lymph node) in a subject. In some embodiments of these methods, the cancer cell metastasis is from a lymph node to a secondary tissue (e.g., any of the secondary tissues described herein) in the subject.

In some embodiments, the cancer cell invasion is the migration of a cancer cell into a tissue proximal to the primary tumor. In some embodiments, the cancer cell invasion is the migration of a cancer cell from a primary tumor into the lymphatic system. In some embodiments, the cancer cell invasion is the migration of a metastatic cancer cell present in the lymph node into the lymphatic system or the migration of a metastatic cancer cell present in a secondary tissue to an adjacent tissue in the subject.

Cancer cell invasion in a subject can be assessed or monitored by visualization using any of the imaging techniques described herein. For example, one or more tissues of a subject having a cancer or metastatic cancer can be visualized at two or more time points (e.g., at a time point shortly after diagnosis with a cancer and at later time point). In some embodiments, a decrease in cancer cell invasion in a subject can be detected by observing a decrease in the spread of a primary tumor through a specific tissue in the subject (when the spread of the primary tumor is assessed through the imaging techniques known in the art or described herein). In some embodiments, a decrease in cancer cell invasion can be detected by a reduction in the number of circulating primary cancer cells or circulating metastatic cancer cells in the blood or lymph of a subject.

Cancer cell metastasis can be detected using any of the methods described herein or known in the art. For example, successful reduction of cancer cell metastasis can be observed as a decrease in the rate of development of an additional metastatic tumor in a subject already having at least one metastatic tumor (e.g., a subject already having a metastatic tumor in a lymph node) (e.g., as compared to the rate of development of an additional metastatic tumor in a subject or a population of subjects already having a similar metastatic tumor but not receiving treatment or receiving an alternative treatment). Successful reduction of cancer cell metastasis can also be observed as a decrease in the risk of developing at least one metastatic cancer (e.g., a metastatic cancer in a lymph node) in a subject having (e.g., diagnosed as having) a primary cancer (e.g., a primary breast cancer) (e.g., as compared to the risk of developing a metastatic cancer in a subject or a population of subjects having a similar primary cancer but not receiving treatment or receiving an alternative treatment).

Methods of Detecting, Diagnosing, and/or Monitoring a Metastatic Cancer Tissue

Also provided herein are methods of detecting, diagnosing, and/or monitoring a metastatic cancer tissue in a subject that include administering any of the therapeutic nanoparticles disclosed herein to the subject having the metastatic cancer tissue and imaging the therapeutic nanoparticle.

In some embodiments of these methods, the therapeutic nanoparticle is administered in an amount sufficient to image the therapeutic nanoparticle in the subject. In some embodiments, the therapeutic nanoparticle amount sufficient to detect, diagnose, and/or monitor a metastatic cancer tissue in a subject is less than about 0.020 mg/kg (e.g., about 0.001 mg/kg to about 0.005 mg/kg, about 0.005 to about 0.010 mg/kg, about 0.010 mg/kg to about 0.015 mg/kg, about 0.011 mg/kg to about 0.015 mg/kg, about 0.012 mg/kg to about 0.015 mg/kg, about 0.013 mg/kg to about 0.015 mg/kg, about 0.012 mg/kg to about 0.016 mg/kg, about 0.013 mg/kg to about 0.017 mg/kg, about 0.013 mg/kg to about 0.018 mg/kg, or about 0.015 mg/kg to about 0.020 mg/kg). In some embodiments, the therapeutic nanoparticle amount sufficient to detect, diagnose, and/or monitor a metastatic cancer tissue in a subject is less than about 0.001 mg/kg. In some embodiments, the therapeutic nanoparticle amount sufficient to detect, diagnose, and/or monitor a metastatic cancer tissue in a subject is less than about 0.014 mg/kg. In some embodiments, the therapeutic nanoparticle amount sufficient to detect, diagnose, and/or monitor a metastatic cancer tissue in a subject does not induce a drug side effect in the subject.

In some embodiments, the imaging is carried out by magnetic resonance imaging (MRI), positron emission tomography (PET), single-photon emission computed tomography (SPECT), computed tomography (CT), or any combination thereof. In some embodiments, the imaging is carried out by PET-MRI. In some embodiments, the therapeutic nanoparticles of the disclosure can accumulate in metastatic cancer tissue and thus, can be effective at highlighting metastatic tissues when imaged (e.g., via PET or PET-MRI).

### Dosing, Administration, and Compositions

In any of the methods described herein, the therapeutic nanoparticle can be administered by a health care professional (e.g., a physician, a physician's assistant, a nurse, or a laboratory or clinic worker), the subject (i.e., self-administration), or a friend or family member of the subject. The administering can be performed in a clinical setting (e.g., at a clinic or a hospital), in an assisted living facility, or at a pharmacy. In some embodiments of any of the methods described herein, the therapeutic nanoparticle is administered to a subject that has been diagnosed as having a cancer (e.g., having a primary cancer or a metastatic cancer). In some embodiments, the subject has been diagnosed with breast cancer (e.g., a metastatic breast cancer). In some non-limiting embodiments, the subject is a man or a woman, an adult, an adolescent, or a child. The subject can have experienced one or more symptoms of a cancer or metastatic cancer (e.g., a metastatic cancer in a lymph node). The subject can also be diagnosed as having a severe or an advanced stage of cancer (e.g., a primary or metastatic cancer). In some embodiments, the subject may have been identified as having a metastatic tumor present in at least one lymph node. In some embodiments, the subject may have already undergone lymphectomy and/or mastectomy.

In some embodiments of any of the methods described herein, the subject is administered at least one (e.g., at least 2, 3, 4, 5, 6, 7, 8, 9, 10, 15, 20, 25, or 30) dose of a composition containing at least one (e.g., one, two, three, or four) of any of the magnetic particles or pharmaceutical compositions described herein. In any of the methods described herein, the at least one magnetic particle or pharmaceutical composition (e.g., any of the magnetic particles or pharmaceutical compositions described herein) can be administered intravenously, intra-arterially, subcutaneously, intraperitoneally, or intramuscularly to the subject. In some embodiments, the at least magnetic particle or pharmaceutical composition is directly administered (injected) into a lymph node in a subject.

In some embodiments, the subject is administered at least one therapeutic nanoparticle or pharmaceutical composition (e.g., any of the therapeutic nanoparticles or pharmaceutical compositions described herein) and at least one additional therapeutic agent. The at least one additional therapeutic agent can be a chemotherapeutic agent (e.g., cyclophosphamide, mechlorethamine, chlorambucil, melphalan, daunorubicin, doxorubicin, epirubicin, idarubicin, mitoxantrone, valrubicin, paclitaxel, docetaxel, etoposide, teniposide, tafluposide, azacitidine, azathioprine, capecitabine, cytarabine, doxifluridine, fluorouracil, gemcitabine, mercaptopurine, methotrexate, tioguanine, bleomycin, carboplatin, cisplatin, oxaliplatin, bortezomib, carfilzomib, salinosporamide A, all-trans retinoic acid, vinblastine, vincristine, vindesine, and vinorelbine) and/or an analgesic (e.g., acetaminophen, diclofenac, diflunisal, etodolac, fenoprofen, flurbiprofen, ibuprofen, indomethacin, ketoprofen, ketorolac, meclofenamate, mefenamic acid, meloxicam, nabumetone, naproxen, oxaprozin, phenylbutazone, piroxicam, sulindac, tolmetin, celecoxib, buprenorphine, butorphanol, codeine, hydrocodone, hydromorphone, levorphanol, meperidine, methadone, morphine, nalbuphine, oxycodone, oxymorphone, pentazocine, propoxyphene, and tramadol).

In some embodiments, at least one additional therapeutic agent and at least one therapeutic nanoparticle (e.g., any of the therapeutic nanoparticles described herein) are administered in the same composition (e.g., the same pharmaceutical composition). In some embodiments, the at least one additional therapeutic agent and the at least one therapeutic nanoparticle are administered to the subject using different routes of administration (e.g., at least one additional therapeutic agent delivered by oral administration and at least one therapeutic nanoparticle delivered by intravenous administration).

In any of the methods described herein, the at least one therapeutic nanoparticle or pharmaceutical composition (e.g., any of the therapeutic nanoparticles or pharmaceutical compositions described herein) and, optionally, at least one additional therapeutic agent can be administered to the subject at least once a week (e.g., once a week, twice a week, three times a week, four times a week, once a day, twice a day, or three times a day). In some embodiments, at least two different therapeutic nanoparticles are administered in the same composition (e.g., a liquid composition). In some embodiments, at least one therapeutic nanoparticle and at least one additional therapeutic agent are administered in the same composition (e.g., a liquid composition). In some embodiments, the at least one therapeutic nanoparticle and the at least one additional therapeutic agent are administered in two different compositions (e.g., a liquid composition containing at least one therapeutic nanoparticle and a solid oral composition containing at least one additional therapeutic agent). In some embodiments, the at least one additional therapeutic agent is administered as a pill, tablet, or capsule. In some embodiments, the at least one additional therapeutic agent is administered in a sustained-release oral formulation.

In some embodiments, the one or more additional therapeutic agents can be administered to the subject prior to administering the at least one therapeutic nanoparticle or pharmaceutical composition (e.g., any of the therapeutic nanoparticles or pharmaceutical compositions described herein). In some embodiments, the one or more additional therapeutic agents can be administered to the subject after administering the at least one therapeutic nanoparticle or pharmaceutical composition (e.g., any of the magnetic particles or pharmaceutical compositions described herein). In some embodiments, the one or more additional therapeutic agents and the at least one therapeutic nanoparticle or pharmaceutical composition (e.g., any of the therapeutic nanoparticles or pharmaceutical compositions described herein) are administered to the subject such that there is an overlap in the bioactive period of the one or more additional therapeutic agents and the at least one therapeutic nanoparticle (e.g., any of the therapeutic nanoparticles described herein) in the subject.

In some embodiments, the subject can be administered the at least one therapeutic nanoparticle or pharmaceutical composition (e.g., any of the therapeutic nanoparticles or pharmaceutical compositions described herein) over an extended period of time (e.g., over a period of at least 1 week, 2 weeks, 3 weeks, 1 month, 2 months, 3 months, 4 months, 5 months, 6 months, 7 months, 8 months, 9 months, 10 months, 11 months, 12 months, 1 year, 2 years, 3 years, 4 years, 5 years, or 10 years). A skilled medical professional may determine the length of the treatment period using any of the methods described herein for diagnosing or following the effectiveness of treatment (e.g., using the methods above and those known in the art). As described herein, a skilled medical professional can also change the identity and number (e.g., increase or decrease) of therapeutic nanoparticles (and/or one or more additional therapeutic agents) administered to the subject and can also adjust (e.g., increase or decrease) the dosage or frequency of administration of at least one therapeutic nanoparticle (and/or one or more additional therapeutic agents) to the subject based on an assessment of the effectiveness of the treatment (e.g., using any of the methods described herein and known in the art). A skilled medical professional can further determine when to discontinue treatment (e.g., for example, when the subject's symptoms are significantly decreased).

### Methods of Preparing the Therapeutic Nanoparticles

Also provided herein are methods of preparing any of the therapeutic nanoparticle of the disclosure that include preparing the magnetic nanoparticle (e.g., via any one of the methods described elsewhere herein), covalently linking the nucleic acid molecule to the magnetic nanoparticle; covalently linking the chelator to the magnetic nanoparticle (MN) by reacting the magnetic nanoparticle with the chelator at a ratio of about 40:1 equivalents (eq.) (i.e., 40 equivalents of the chelator to 1 equivalent of the magnetic nanoparticle), adding a solution of ⁶⁴CuCl₂ to the magnetic nanoparticle, and purifying a mixture of the solution of ⁶⁴CuCl₂ and the magnetic nanoparticle to yield the therapeutic nanoparticle.

In some embodiments, the methods include reacting the magnetic nanoparticle with the chelator at a ratio ranging from about 5:1 chelator eq.:MN to about 60:1 chelator eq.:MN (e.g., about 5:1 chelator eq.:MN to about 10:1 chelator eq.:MN, about 5:1 chelator eq.:MN to about 12:1 chelator eq.:MN, about 5:1 chelator eq.:MN to about 15:1 chelator eq.:MN, about 5:1 chelator eq.:MN to about 20:1 chelator eq.:MN, about 5:1 chelator eq.:MN to about 25:1 chelator eq.:MN, about 5:1 chelator eq.:MN to about 30:1 chelator eq.:MN, about 5:1 chelator eq.:MN to about 35:1 chelator eq.:MN, about 5:1 chelator eq.:MN to about 40:1 chelator eq.:MN, about 5:1 chelator eq.:MN to about 45:1 chelator eq.:MN, about 5:1 chelator eq.:MN to about 50:1 chelator eq.:MN, about 5:1 chelator eq.:MN to about 55:1 chelator eq.:MN, or about 5:1 chelator eq.:MN to about chelator eq.:MN, about 10:1 chelator eq.:MN to about 12:1 chelator eq.:MN, about chelator eq.:MN to about 15:1 chelator eq.:MN, about 10:1 chelator eq.:MN to about chelator eq.:MN, about 10:1 chelator eq.:MN to about 25:1 chelator eq.:MN, about 10:1 chelator eq.:MN to about 30:1 chelator eq.:MN, about 10:1 chelator eq.:MN to about chelator eq.:MN, about 10:1 chelator eq.:MN to about 40:1 chelator eq.:MN, about chelator eq.:MN to about 45:1 chelator eq.:MN, about 10:1 chelator eq.:MN to about chelator eq.:MN, about 10:1 chelator eq.:MN to about 55:1 chelator eq.:MN, or about 10:1 chelator eq.:MN to about 60:1 chelator eq.:MN, about 15:1 chelator eq.:MN to about 20:1 chelator eq.:MN, about 15:1 chelator eq.:MN to about 25:1 chelator eq.:MN, about 15:1 chelator eq.:MN to about 30:1 chelator eq.:MN, about 15:1 chelator eq.:MN to about 35:1 chelator eq.:MN, about 15:1 chelator eq.:MN to about 40:1 chelator eq.:MN, about 15:1 chelator eq.:MN to about 45:1 chelator eq.:MN, about 15:1 chelator eq.:MN to about 50:1 chelator eq.:MN, about 15:1 chelator eq.:MN to about 55:1 chelator eq.:MN, or about 15:1 chelator eq.:MN to about 60:1 chelator eq.:MN, about 20:1 chelator eq.:MN to about 45:1 chelator eq.:MN, about 25:1 chelator eq.:MN to about 45:1 chelator eq.:MN, about 30:1 chelator eq.:MN to about 45:1 chelator eq.:MN, about 35:1 chelator eq.:MN to about 45:1 chelator eq.:MN, about 40:1 chelator eq.:MN to about 45:1 chelator eq.:MN, about 35:1 chelator eq.:MN to about 50:1 chelator eq.:MN, about 38:1 chelator eq.:MN to about 40:1 chelator eq.:MN, about 38:1 chelator eq.:MN to about 42:1 chelator eq.:MN, about 38:1 chelator eq.:MN to about 45:1 chelator eq.:MN, about 40:1 chelator eq.:MN to about 50:1 chelator eq.:MN, about 40:1 chelator eq.:MN to about 55:1 chelator eq.:MN, or about 40:1 chelator eq.:MN to about 60:1 chelator eq.:MN).

In some embodiments, covalently linking the chelator to the magnetic nanoparticle is performed at a temperature of about 0° C. to about 8° C. (e.g., about 0° C. to about 4° C., about 1° C. to about 4° C., about 2° C. to about 4° C., about 3° C. to about 4° C., about 2° C. to about 6° C., about 3° C. to about 7° C., about 4° C. to about 5° C., about 4° C. to about 6° C., about 4° C. to about 7° C., or about 4° C. to about 8° C.). In some embodiments, covalently linking the chelator to the magnetic nanoparticle is performed at a temperature of about 4° C.

In some embodiments, these methods further include heating the mixture of the solution of ⁶⁴CuCl₂ and the magnetic nanoparticle at a temperature of about 40° C. to about 65° C. (e.g., about 40° C. to about 65° C., about 45° C. to about 65° C., about 50° C. to about 65° C., about 55° C. to about 65° C., about 56° C. to about 65° C., about 57° C. to about 65° C., about 58° C. to about 65° C., about 59° C. to about 65° C., about 58° C. to about 62° C., about 59° C. to about 61° C., about 58° C. to about 63° C., about 59° C. to about 63° C., about 59° C. to about 60° C., about 60° C. to about 61° C., or about 60° C. to about 62° C.). In some embodiments, these methods further include heating the mixture of the solution of ⁶⁴CuCl₂ and the magnetic nanoparticle at a temperature of about 60° C. In some embodiments, these methods further include heating the mixture of the solution of ⁶⁴CuCl₂ and the magnetic nanoparticle for about 10 min. to about 30 min. (e.g., about 10 min. to about 20 min., about 11 min. to about 21 min., about 12 min. to about 22 min., about 13 min. to about 23 min., about 14 min. to about 24 min., about 15 min. to about 25 min., about 16 min. to about 26 min., about 13 min. to about 20 min., about 15 min. to about 20 min., about 15 min. to about 22 min., about 18 min. to about 20 min., about 18 min. to about 22 min., about 19 min. to about 21 min., about 19 min. to about 23 min., about 20 min. to about 25 min., or about 20 min. to about 30 min.). In some embodiments, these methods further include heating the mixture of the solution of ⁶⁴CuCl₂ and the magnetic nanoparticle for about 20 min.

In some embodiments, these methods do not subject the therapeutic nanoparticles to temperatures exceeding about 65° C. In some embodiments, these methods do not subject the therapeutic nanoparticles to temperatures exceeding about 60° C. In some embodiments, the methods disclosed herein including the use of a chelator to associate the radiolabel with the therapeutic nanoparticle advantageously avoid any harsh conditions (e.g., temperatures exceeding about 60° C. for longer than about 20 minutes) that may potentially damage the nucleic acid molecule.

### EXAMPLES

Certain embodiments of the present disclosure are further described in the following examples, which do not limit the scope of any embodiments described in the claims.

### Example 1. In Vitro Studies of MN-Anti-miR10b

Twenty to thirty-five nm dextran-coated therapeutic magnetic nanoparticles were functionalized with antisense locked nucleic acid (LNA) oligonucleotides targeting human miRNA-10b. The resulting therapeutic magnetic nanoparticles (MN) are hereafter referred to as “MN-anti-miR-10b.” In vitro cell studies testing these therapeutic magnetic nanoparticles were performed as described below.

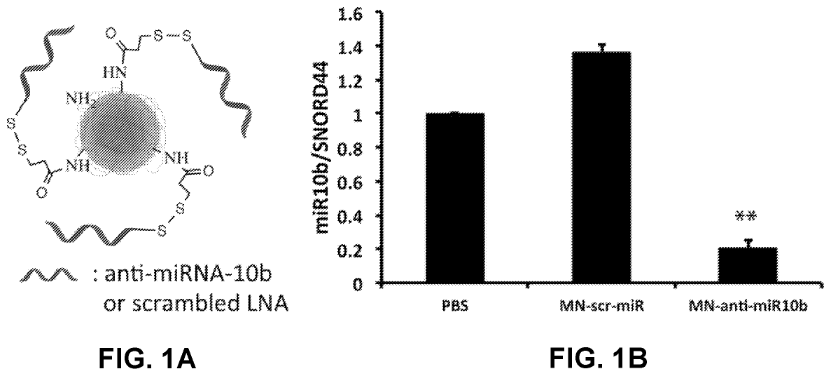

21.6±0.5-nm dextran coated magnetic nanoparticles were conjugated to 8±0.7 LNA antagomirs targeting miRNA-10b, (Exiqon, Woburn, MA) (FIG. 1A). The MN-anti-miR10b therapeutic was functional in the human lymph-node metastatic MDA-MB-231-luc-D3H2LN cell line (Perkin Elmer, Hopkinton MA). The peak of efficacy was achieved after a 48-h incubation with MN-anti-miR10b (0.7 nmoles ASO/ml) at which point there was a significant 86.3±5.8% inhibition of the target miRNA-10b, compared to the inactive therapeutic MN-scr-miR functionalized with an irrelevant oligonucleotide (Exiqon, Woburn, MA) (FIG. 1B). These results indicated that the delivery method and therapeutic design could be used to very efficiently inhibit miRNA-10b in tumor cells. In order to assess the therapeutic potential of this methodology, the effect of miR-10b inhibition on tumor cell apoptosis, proliferation, invasion and migration was investigated.

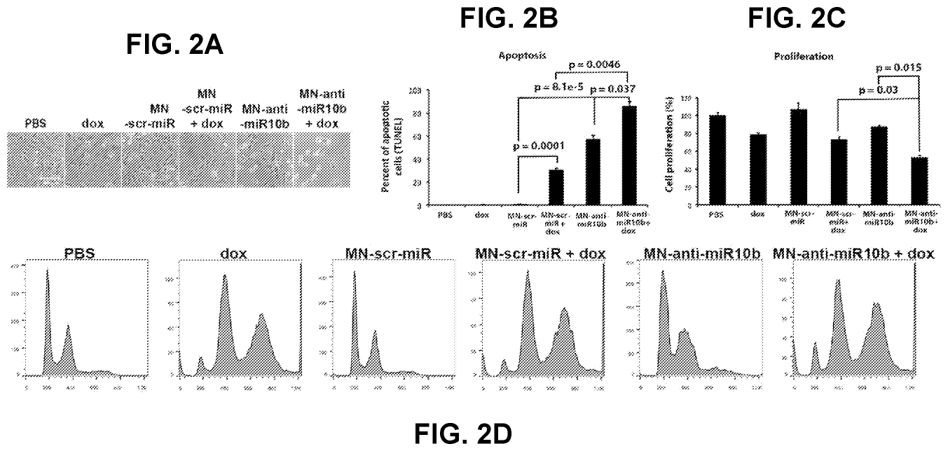

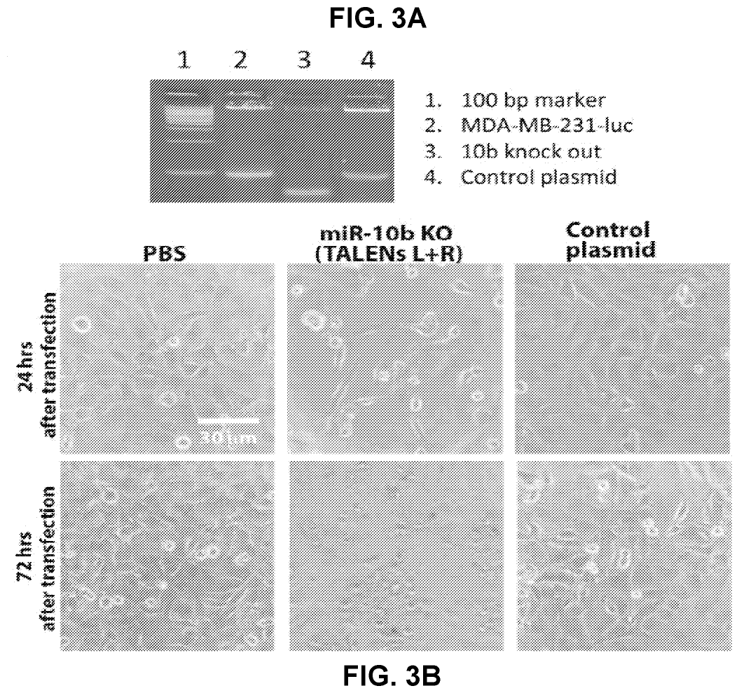

It was found that miR-10b inhibition by the therapeutic led to a significant induction of apoptosis, inhibition of proliferation, and reduction in invasion and migration (FIG. 2). It was also hypothesized that by co-administering a low dose of a cytotoxic chemotherapeutic (e.g., doxorubicin), the effectiveness of the therapeutic could be amplified, leading not only to inhibition of tumor growth, but regression and, possibly, elimination of metastasis. It was observed that following combination treatment with the therapeutic (0.5 μM) and a low-dose of doxorubicin (0.05 μM, <IC10), the tumor cells acquired a spindle-like shape, detached, and died (FIG. 2A). This corresponded to a significant induction of apoptosis (FIG. 2B) and decrease in proliferation (FIG. 2C). Analysis of the effect of the combination treatment on the cell cycle revealed a potential mechanism underlying the observed synergy between doxorubicin and the therapeutic. Namely, doxorubicin alone or in combination with the therapeutic induced polyploidy and G2/M arrest (FIG. 2D). This indicated that in the described scenario, doxorubicin, by inhibiting the cell cycle and reducing the rate of cell division in the tumor cells, amplified the pro-apoptotic effect of the MN-anti-miR10b therapeutic, likely through ensuring a higher local concentration of the therapeutic. In line with this hypothesis, when low-dose doxorubicin and MN-anti-miR10b were combined, there was an increase of both the pre-G1 apoptotic and G2/M arrested cell populations (FIG. 2D). These studies indicated that by combining a low dose cytostatic, such as doxorubicin, and miR-10b inhibition using the described therapeutic, it is possible to mediate a profound effect on tumor cell phenotype manifested at its endpoint by tumor cell death and loss of invasive properties. These results also suggested that miR-10b is a metastamir essential for tumor cell survival, akin to the mechanism behind oncomir addiction. Further proof for this hypothesis came from experiments in which we attempted to generate a miR-10b knock-out subline derived from the MDA-MB-231-luc-D3H2LN cell line (FIG. 3A). We observed that within 24 h of transfection with the knock-out vector set (TALEN5 L+R), the cells acquired a spindle-shaped conformation and began to detach. By 72 h of transfection, there were no viable cells (FIG. 3B). This latter result suggested that a robust therapeutic effect could possibly be achieved by treatment with a higher dose of the MN-anti-miR10b therapeutic alone, even in the absence of doxorubicin.

### Example 2. The MN-Anti-miR10b Therapeutic can be Delivered to Distant Metastatic Tumor Cells In Vivo

Having established in vitro that targeting of miR-10b using the therapeutic nanoparticles and a low-dose chemotherapeutic could have profound implications in a therapeutic scenario, it was determined if the observed therapeutic effect could be achieved in vivo. The first step was to assess whether the therapeutic could be delivered to the target metastatic organs.

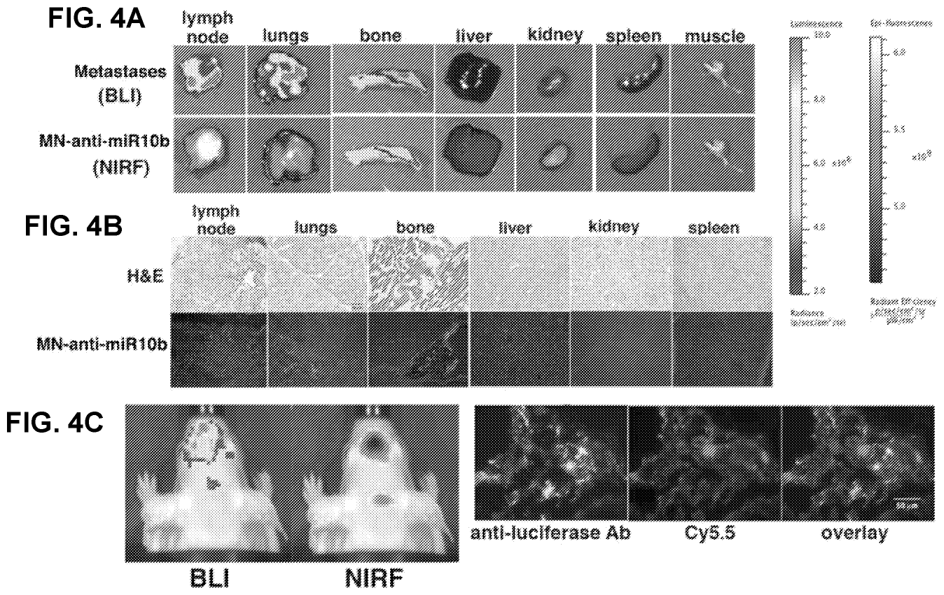

Fluorescently labeled therapeutic (labeled with Cy5.5 on MN) was injected into balb/c mice implanted orthotopically with the murine breast adenocarcinoma 4T1-luc2 cell line. In this model, orthotopically-implanted tumors progress from localized disease to lymph node, lung, and bone metastases by 3 weeks after tumor inoculation. Ex vivo near infrared (NIRF) optical imaging performed 24 h after intravenous injection of the therapeutic revealed uptake by the metastatic lesions in the lymph nodes, lungs, and bone (FIG. 4A). Fluorescence microscopy confirmed widespread uptake by the metastatic tumor cells in these organs (FIG. 4B) supporting our hypothesis that the therapeutic, as designed can target disseminated cancer to distant organs.

Low level accumulation of MN-anti-miR10b in the organs of the reticuloendothelial system was expected. There, it was taken up by the cells and rapidly broken down. The iron from the iron oxide core entered the endogenous iron pool, whereas the dextran from the nanoparticle coating was cleared through the kidneys. In vivo imaging in animals with established brain metastases (FIG. 4C) was performed and uptake by the metastatic lesions was demonstrated. This suggested that the degree of disruption of the blood-brain barrier in this model was sufficient to permit delivery of the therapeutic.

### Example 3. Therapy with MN-Anti-miR10b can Trigger Regression of Lymph Node Metastases

With an outlook towards clinical translation of our therapeutic approach, low-dose doxorubicin (4 mg/kg weekly i.p.) and the MN-anti-miR10b therapeutic (15 mg/kg Fe, 10 mg/kg ASO weekly i.v.) was combined with the goal of regressing lymph node metastatic breast cancer. Specifically, 4-5 weeks after tumor inoculation, mice bearing orthotopic MDA-MB-231-luc-D3H2LN tumors had their primary tumors surgically removed following confirmation of lymph node metastasis. This was done to better simulate a clinical scenario, since the current standard of care involves surgical removal of the primary tumor in patients with lymph node metastatic breast cancer.

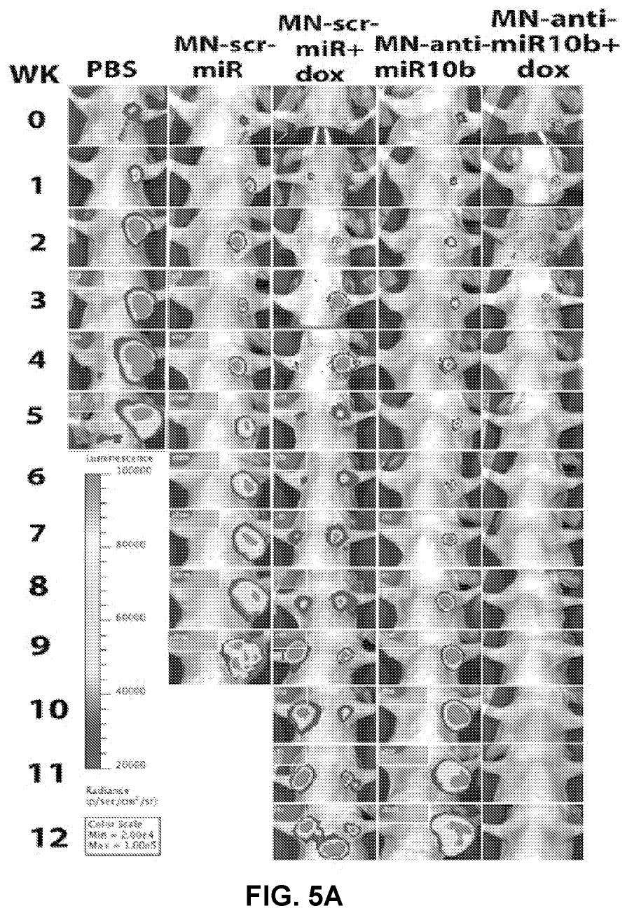

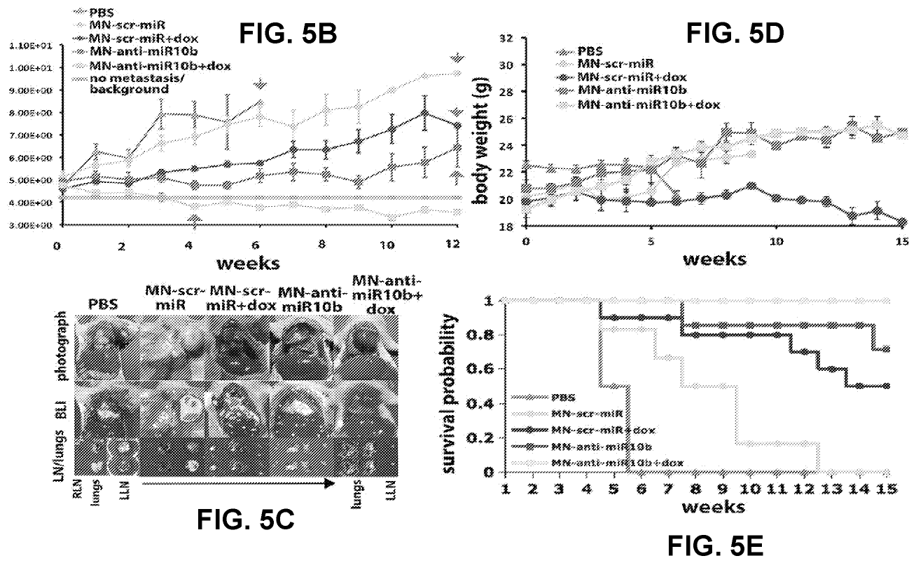

Treatment was initiated on the week of tumor removal. There was complete regression of lymph node metastases in the experimental mice treated with MN-antimiR10b and low-dose doxorubicin after 4 weeks of therapy (FIGS. 5A and 5B). By contrast, in the control groups, there was metastatic progression. In the experimental mice, treatment was discontinued once complete metastatic regression was observed at week 4. Still, by the endpoint of the study, no recurrence was observed (FIGS. 5A and 5B).

Ex vivo analysis revealed the presence of lymph node metastases and metastatic dissemination to the lungs in control animals treated with PBS or inactive therapeutic with or without doxorubicin (FIG. 5C). In the mice treated with MN-anti-miR10b only, there was evidence of metastasis to the lymph nodes but not to the lungs. In contrast, in mice treated with MN-antimiR10b and doxorubicin no gross lymph node or lung metastases could be detected (FIG. 5C).

The effect of therapy with MN-anti-miR10b and low-dose doxorubicin also translated into significant improvement in body weight (FIG. 5D) and survival (FIG. Unlike control groups, by week 14 after the beginning of therapy, none of the experimental animals (MN-anti-miR10b/doxorubicin) had succumbed to cancer. Furthermore, combination therapy with doxorubicin and MN-anti-miR10b was superior to monotherapy with the therapeutic (FIGS. 5A, 5B, 5C, and 5E).

By the 15^{th} week of therapy, the differences between experimental and control animals were apparent by simple anatomical observation. The control animals treated with MN-scr-miR and doxorubicin displayed gross lymphadenopathy and cachexia. The experimental animals treated with MN-anti-miR10b and doxorubicin appeared healthy. Long-term observation indicated that the remission of metastatic disease was permanent and that the treatment resulted in an effective cure for the natural life of the animals. Having demonstrated efficacy, the important issue of systemic toxicity needed to be addressed. A panel of blood chemistry markers including indicators of renal, liver toxicity, etc. was analyzed and no significant effects that could be assigned to components of the therapeutic were found. Histopathology of major organs demonstrated the absence of gross tissue abnormalities.

### Example 4—Therapy with MN-Anti-miR10b can Trigger Regression of Distant Metastases

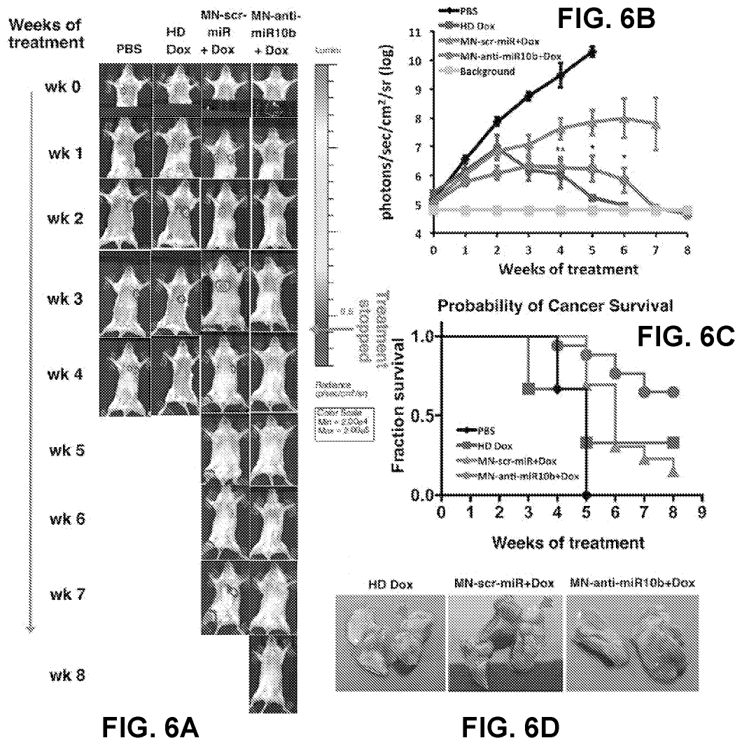

With specific relevance to distant metastases, low-dose doxorubicin (2 mg/kg weekly i.p.) and the MN-antimiR10b therapeutic (15 mg/kg Fe, 10 mg/kg ASO weekly i.v.) was combined in immunocompetent mice (Balb/c) bearing orthotopic 4T1-luc2 breast tumors. In control mice treated with PBS, metastases progressed rapidly (FIGS. 6A and 6B), and resulted in 100% animal mortality by week 5 (FIG. 6C). In mice treated with MN-scr-miR+dox, metastases grew more slowly than in the PBS controls (FIGS. 6A and 6B). There was an 80% cancer mortality by week 7 (FIGS. 6A and 6B). By contrast, in the mice treated with MN-anti-miR10b+dox, we observed regression of distant metastases, evident by week 6 (FIGS. 6A and 6B) at which point treatment was stopped (red arrow in FIG. 6A). The animals remained metastasis-free for the course of the experiment. Regression was accomplished in 65% of the animals, whereas 35% of the animals progressed, due to inefficient inhibition of miR-10b in this group (FIG. 6C). Macroscopic observation of the lungs post-necropsy confirmed the absence of metastatic lesions in the responders treated with MN-anti-miR10b+dox (FIG. 6D).

### Example 5. Synthesis and Characterization of ^{nat/64}Cu-MN-Anti-miR10b

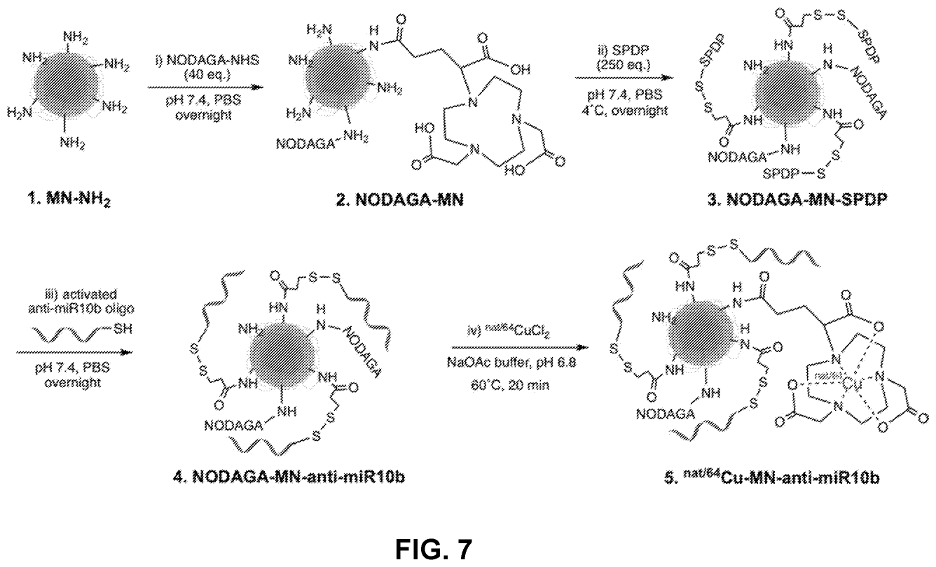

The synthesis of anti-miR10b and ultrasmall iron oxide magnetic nanoparticles (MN) radiolabeled with Cu-64 (64 Cu), hereafter referred to as “64 Cu-MN-anti-miR10b,” started with the modification of MN, a 20-nm aminated dextran-coated iron oxide nanoparticle whose synthesis was previously reported (Yoo et al., 2014). The nanoparticles were optimized to enhance the extravasation of the agent into the interstitium of tumors and metastatic lesions (Yoo et al., 2017a). MN nanoparticles were functionalized with NODAGA, a chelating ligand, by a coupling reaction between the amino groups and the activated ester moiety of NODAGA (FIG. 7). The NODAGA chelator has the ability to rapidly form highly stable ⁶⁴Cu complexes, essential for preventing in vivo dissociation of the radiometal and its subsequent retention in the body. The number of NODAGA chelators per nanoparticle was quantified as 13±2. The ratio of Cu/nanoparticle was determined as 14±1 by ICP-MS after complex formation using ^{nat}CuCl₂. Prior to in vivo studies, the nanoparticles were treated with SPDP and functionalized with anti-miR-10b antagomirs via a disulfide linkage. The number of antagomirs per nanoparticle was characterized as 7.4±0.2 following previously described procedures (Yoo et al., 2014; Yoo et al., 2015). These therapeutic magnetic, radiolabeled nanoparticles were generated and characterized as described below. All reactants and reagents were of commercial grade and were used without further purification. All solutions were prepared from MilliQ water. Metal-free buffer solutions used for radiolabeling were prepared using Chelex 100 Resin (100-200 mesh, BioRad).

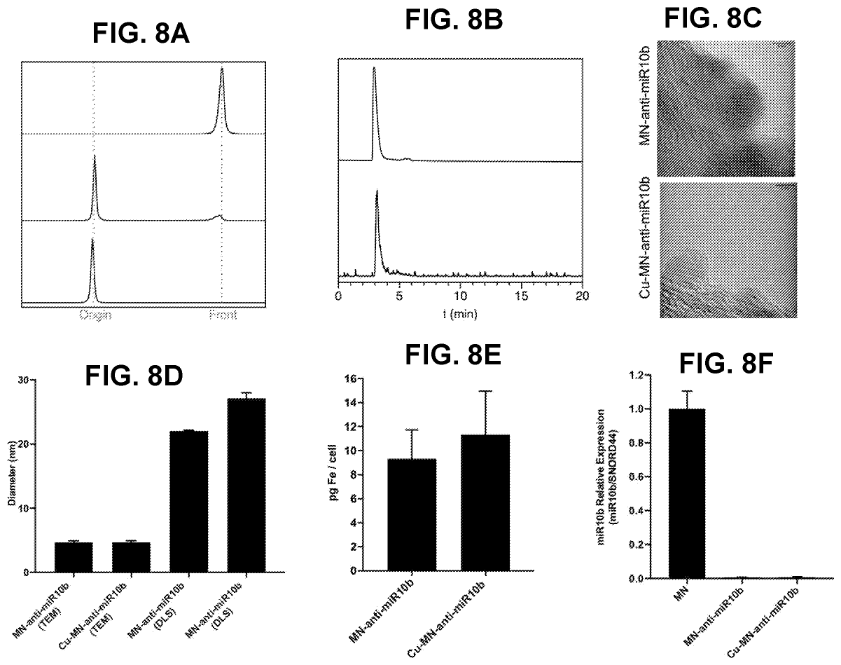

Evaluating the in vivo biodistribution of ⁶⁴Cu-MN-anti-miR10b requires the development of efficient labeling and purification methods. The radiolabeling of MN-anti-miR10b was achieved in acetate buffer at pH 6.8, 60° C. for 20 mins. These conditions are favorable for the labeling of NODAGA chelators while maintaining the integrity of the nanocarrier. After purification on a PD-10 column, ⁶⁴Cu-MN-anti-miR10b was obtained with a radiochemical purity>99% (FIG. 8A) with a specific activity of 7.1 mCi/mg of iron. Radiochemical identity was confirmed by size-exclusion chromatography (FIG. 8B). In addition, ^{nat}Cu-MN-anti-miR10b was synthesized to evaluate the effect of the presence of Cu-NODAGA chelates on the nanoparticle size and target engagement. The size of the iron oxide crystals in the core was measured as 4.71±0.24 nm for ^{nat}Cu-MN-anti-miR10b and 4.69±0.23 nm for MN. The surface modifications did not cause any significant changes in the size and crystal structure of the iron oxide core as shown by transmission electron microscopy (TEM, FIG. 8C). The hydrodynamic diameter of the dextran coated functionalized nanoparticles, anti-miR10b, was determined as 27.1±0.9 nm, which is 5.1 nm larger than that of parent MN. The introduction of Cu-NODAGA and the anti-miR10b antagomir resulted in a 23% increase in hydrodynamic diameter (FIG. 8D).

The cellular uptake, expressed as elemental iron per cell was 9.33±2.42 pg Fe per cell for MN-anti-miR10b and 11.34±3.62 pg Fe per cell for ^{nat}Cu-MN-anti-miR10b, which was not significantly different (FIG. 8E). Finally, the RNA-enriched cell extracts were analyzed to compare the inhibition of miR10b by qRT-PCR. Compared with the expression level of miR10b after treatment with parent MN devoid of antagomir, miR10b expression was completely inhibited following treatment with MN-anti-miR10b or ^{nat}Cu-MN-anti-miR10b (FIG. 8F).

### Synthesis of NODAGA-MN-Anti-miR10b

The steps for the synthesis of ^{nat/64}Cu-MN-anti-miR10b are outlined in FIG. 7. Amine-derivatized iron oxide nanoparticles (MN) were prepared from dextran-coated iron oxide nanoparticles through modification with epichlorohydrin and ammonium hydroxide as described previously (Yoo et al., 2014). The nanoparticles were conjugated with NODAGA-NHS (Chematech, France) by reacting 1 ml of MN (87 μM, 10 mg Fe/ml, 54 NH₂/MN) with 2.54 mg of NODAGA-NHS ester (3.47 μmol, 40 eq. to MN) in 100 μl PBS buffer (100 mM, pH 7.4). The reaction was carried out overnight at 4° C., then the resulting NODAGA-conjugated nanoparticles (NODAGA-MN) were purified with a size exclusion column (PD-10, GE Healthcare) using nuclease free PBS buffer as an eluent. NODAGA-MN was treated with excess amounts of SPDP (250 eq.) for 4 hrs at 4° C. to form NODAGA-MN-SPDP, which was again purified with a size exclusion column using nuclease free PBS buffer as an eluent. The LNA antagomir, anti-miR10b is synthesized and provided by Biospring (Frankfurt, Germany) following a GLP protocol. The anti-miR10b LNA antagomir was modified with the 5′-Thiol-Modifier C6 disulfide (5′-ThioMC6), which was utilized for conjugation to MN. The disulfide on the oligonucleotide was activated by 3% Tris (2-carboxyethyl) phosphine hydrochloride (TCEP, Thermo Scientific Co.), followed by purification with ammonium acetate/ethanol precipitation treatment prior to conjugation to MN. After TCEP activation and purification, the oligo was dissolved in nuclease free water and incubated with NODAGA-MN-SPDP overnight. The final product, NODAGA-MN-anti-miR10b, was freshly prepared prior to animal studies.

Preparation of Non-Radioactive ^{nat}Cu-MN-Anti-miR10b

Nonradioactive ^{nat}Cu-MN-anti-miR10b was prepared to evaluate the inhibitory effect of miR-10b in 4T1 cells. 0.6 mg Fe of NODAGA-MN-anti-miR10b were dispersed in acetate buffer (500 μL, pH 6.8, 0.1 M) followed by the addition of CuCl₂ (0.7 mg, 50 equiv. Cu ²⁺ to NODAGA). The reaction mixture was stirred at 60° C. for 20 min and EDTA (100 μL, 100 mM, pH 7.4) was added to the mixture to remove any unlabeled free Cu ²⁺ ions followed by purification with size exclusion column (PD-10, GE Healthcare) using nuclease free PBS buffer as an eluent. Fractions containing the desired product were combined and the concentration of ^{nat}Cu-MN-anti-miR10b was determined by ICP-MS.

### Preparation of Radiolabeled MN-Anti-miR10b (64 Cu-MN-Anti-miR10b)

Radiolabeling was performed following commonly used procedures (Desogere et al., 2017). Briefly, 200 μg (as Fe) of NODAGA-MN-anti-miR10b in PBS was added to a solution of ⁶⁴CuCl₂ (4 mCi, 148 MBq, the University of Wisconsin at Madison, WI) in sodium acetate buffer (0.1 M, pH 6.8, 500 μL). The reaction mixture was heated at 60° C. for 20 min and then purified with a size exclusion column (PD-10 column) using nuclease free PBS buffer as an eluent and each 500 μL of eluent was collected as a fraction. The radiochemical purity of each fraction was controlled by iTLC (Agilent, iTLC-SG, Santa Clara, CA) with an EDTA solution as an eluent (50 mA/1, pH 5) using a radio-TLC imaging scanner (AR-2000, Eckert & Ziegler, Berlin, Germany). Fractions with a radiochemical purity>99% were combined and used for in vivo animal studies. Radiochemical identity of the final solution of ⁶⁴Cu-MN-anti-miR10b was confirmed by analytical HPLC (Agilent 1100 HPLC system, Santa Clara, CA) with a size exclusion column (TSK gel QC-PAK-300, isocratic, 100% sodium phosphate 0.1 M pH 7.4, 20 min) and a Carroll/Ramsey radioactivity detector with a silicon PIN photodiode and with UV detection at 254 nm.

Characterization of MN-Anti-miR10b and natCu-MN-Anti-miR10b

Inductively Coupled Plasma Mass Spectrometry (ICP-MS) analysis (Agilent 8800-QQQ system, Santa Clara, CA) was carried out to determine the concentrations of copper and iron. All samples were prepared by weight. Calibration standards were prepared by diluting certified copper and iron standards (1000 mg/L). Calibration curve was obtained from 5 standard solutions in the range from 0.1 to 400 ppb. Lutetium (1 ppm) was used as an external standard to ensure the proper introduction of the sample. The hydrodynamic diameter and Zeta-potential were measured by a dynamic light scattering spectrometer (Zetasizer Nano, Malvern, UK) and the size of the iron oxide core was determined by transmission electron microscopy (JEM 2100 TEM, Jeol, Tokyo, Japan). To quantify the number of NODAGA per MN, the number of amines per MN was subtracted from the number of amines per MN after conjugation with NODAGA. The number of amines per MN was quantified by pyridine-2-thione (343 nm, 8080 M⁻¹cm⁻¹) released from SPDP that was conjugated to the amine groups at a one-to-one ratio (ThermoFisher, Waltham, MA). Finally, the number of oligonucleotides per MN was determined by spectrophotometry with multiple standards of different concentrations. Briefly, MN-anti-miR10b and ^{nat}Cu-MN-anti-miR10b were purified using a magnetic column (MACS column, Miltenyi, Cambridge, MA) to remove unbound anti-miR10b oligo. The purified nanoparticles were assayed to determine iron concentration (410 nm) and the concentration of oligo (260 nm) by spectrophotometry (Spectramax M2 microplate reader, Molecular Devices, Sunnyvale, CA).

### Preparation and Characterization of Radiolabeled ⁸⁹Zr-MN-Anti-miR10b

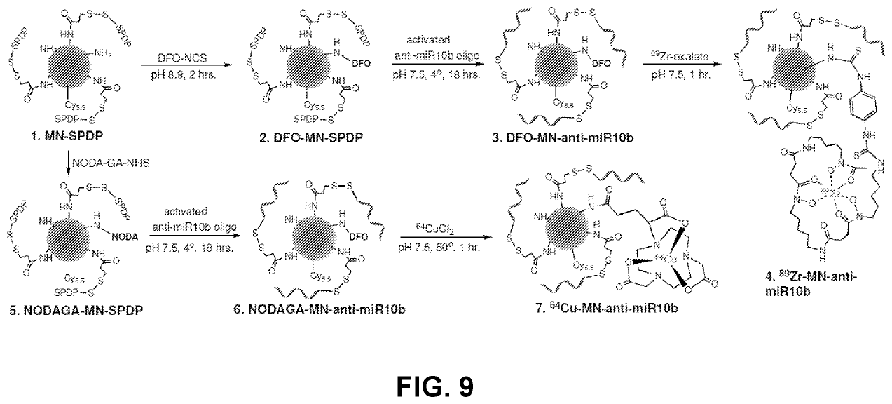

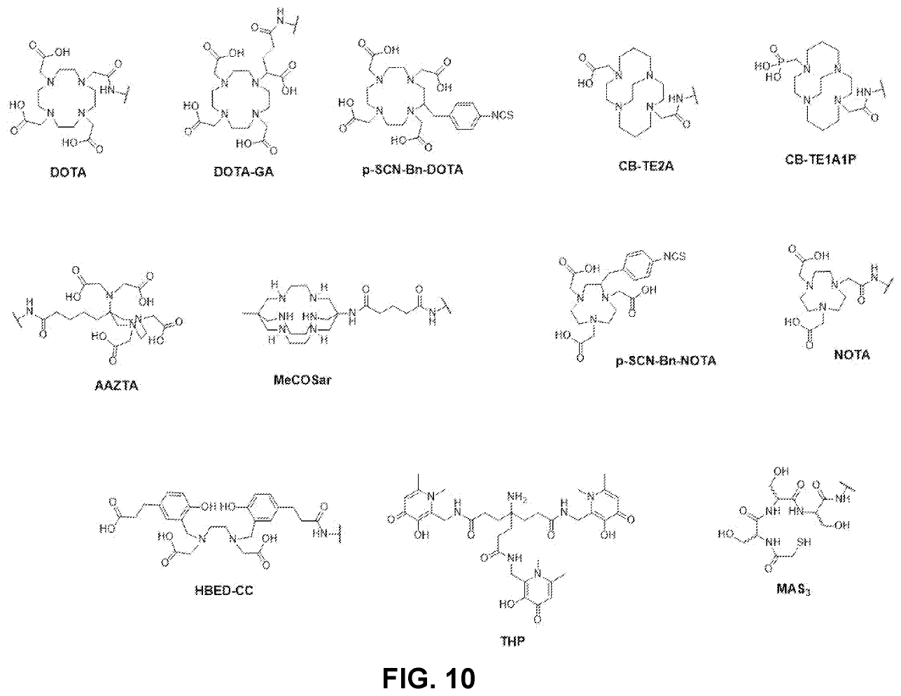

The steps for the synthesis of ⁸⁹Zr-MN-anti-miR10b are outlined in FIG. 9. The isothiocyanate derivative of deferoxamine (DFO) (DFO-Bz-NCS, Macrocyclics, Plano, TX) were conjugated to an amine group on MN to form thiourea bonding (12, 13). MN-anti-miR10b (5 mg/mL) was dispersed into 0.1 M NaHCO₃(containing 0.9% NaCl, pH 8.9) and reacted with a 5-fold molar excess of DFO-Bz-NCS (DFO-NCS, 1 mg/mL in DMSO). The mixture was placed on a rocker at 37° for 2 hrs and 1 M Tris was added to terminate the conjugation reaction (final concentration of Tris:15 mM). The characterization of ⁸⁹Zr-MN-anti-miR10b followed the same steps for the characterization of MN-anti-miR10b and ^{nat}Cu-MN-anti-miR10b outlined above. For example, to quantify the number of DFO per MN, the number of amines per MN was subtracted from the number of amines per MN after conjugation with DFO. FIG. 10 shows additional, example chelators that can be suitable for synthesis of the therapeutic magnetic, radiolabeled nanoparticles described herein.

### Cellular Uptake

The cellular uptake of ^{nat}Cu-MN-anti-miR10b was compared with that of MN-anti-miR10b and parent MN. 4T1-luc cells were seeded in a 12-well plate and incubated with ^{nat}Cu-MN-anti-miR10b, MN-anti-miR10b, and MN for 24 hrs at 37° C. After washing with DPBS, the cells were lysed (Cell lysis buffer, Sigma-Aldrich, St. Louis, MO) and analyzed by ICP-MS to determine the concentration of iron. The protein concentration was determined by BCA assay (Sigma-Aldrich, St. Louis, MO). The cellular uptake of nanoparticles was normalized by total protein.

### Real-Time Quantitative Reverse Transcription-PCR

To assess target engagement by ^{nat}Cu-MN-anti-miR10b as compared to the unlabeled MN-anti-miR10b, 4T1-luc cells were incubated with ^{nat}Cu-MN-anti-miR10b, MN-anti-miR10b, and MN for 48 hrs at 37° C. From the cell lysates, the microRNA-enriched fraction was harvested using a miRNeasy mini kit following the manufacturer's protocol (Qiagen Inc., Hilden, Germany). Relative expression of miR-10b was determined by real-time quantitative reverse transcription-PCR (qRT-PCR; Taqman protocol) and normalized to the internal housekeeping gene, SNORD44. Taqman analysis was carried out using an ABI Prism 7700 sequence detection system (Applied Biosystems, Foster City, CA). The primers (Hs-miR-10b-3 miScript Primer, Hs-SNORD44-11 miScript Primer) and assay kit (miScript PCR Starter Kit, Qiagen, Hilden, Germany).

### Example 6. Biodistribution of ⁶⁴Cu-MN-Anti-miR10b

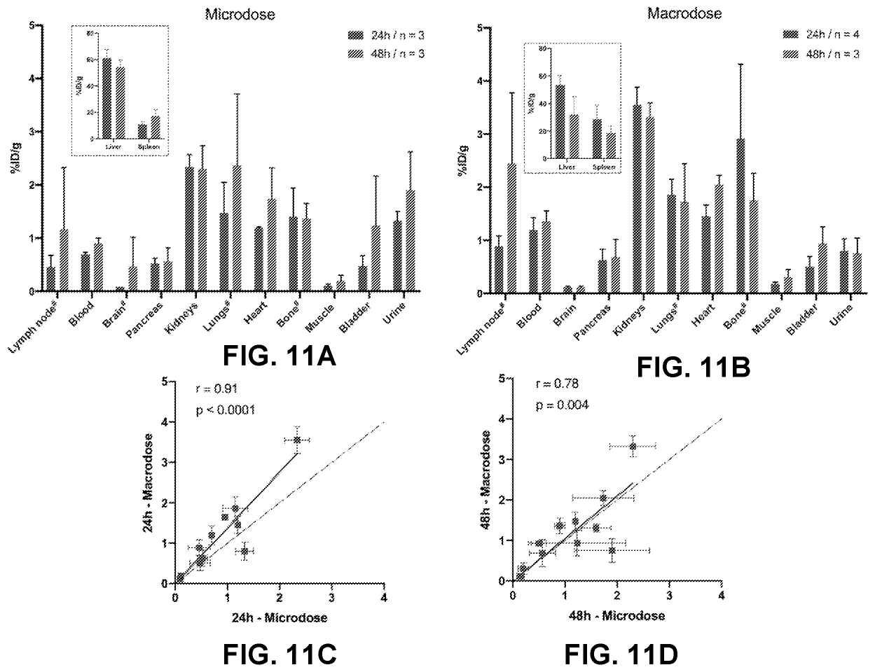

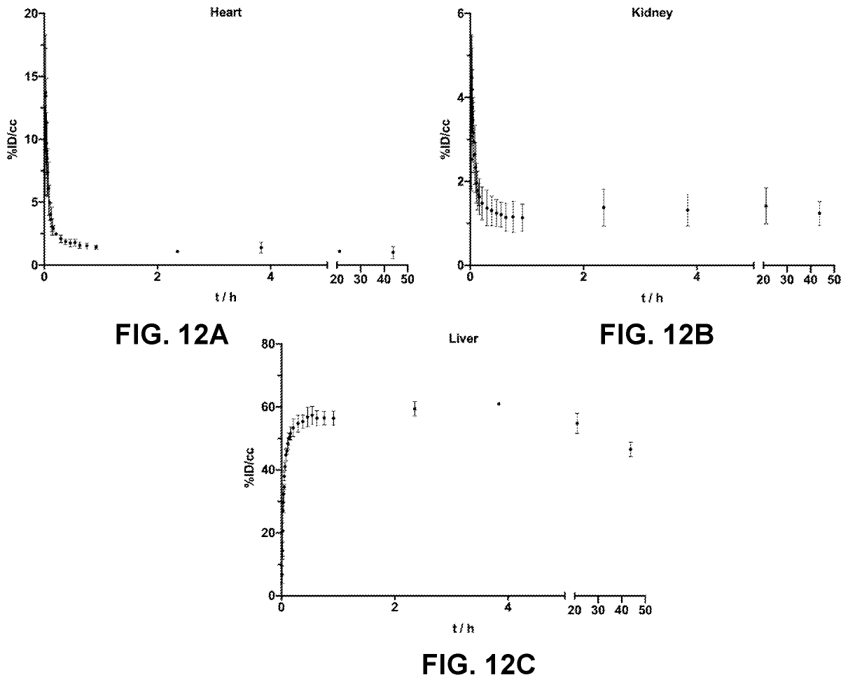

Next, the biodistribution of ⁶⁴Cu-MN-anti-miR10b was evaluated by PET-MRI and ex vivo gamma counting in a total of 13 mice bearing luciferase-expressing metastatic breast adenocarcinomas (4T1-luc2). 4T1-luc2 cells express luciferase and can be detected by non-invasive bioluminescence imaging (BLI). Two different concentrations of ⁶⁴Cu-MN-anti-miR10b were investigated: 1) a tracer level dose of ⁶⁴Cu-MN-anti-miR10b, referred to as microdose (1 mg Fe/kg, n=6), and 2) a therapeutic dose, referred to as macrodose, corresponding to 64 Cu-MN-anti-miR10b co-injected with MN-anti-miR10b (15 mg Fe/kg, n=7). Mice were scanned by PET-MRI at different time points. After in vivo imaging, the mice were sacrificed at 24 h p.i. (n=3 in the microdose group and n=4 in the macrodose group) and 48 h p.i. (n=3 in each group) for ex vivo biodistribution evaluation (FIGS. 11A-11D). Time-activity curves for liver, kidney and heart obtained from the PET images (FIGS. 12A-12C) indicate that most of the injected dose is rapidly taken up by the liver. This is in line with previously reported studies (Briley-Saebo et al., 2004; Estevanato et al., 2012; Schlachter et al., 2011).

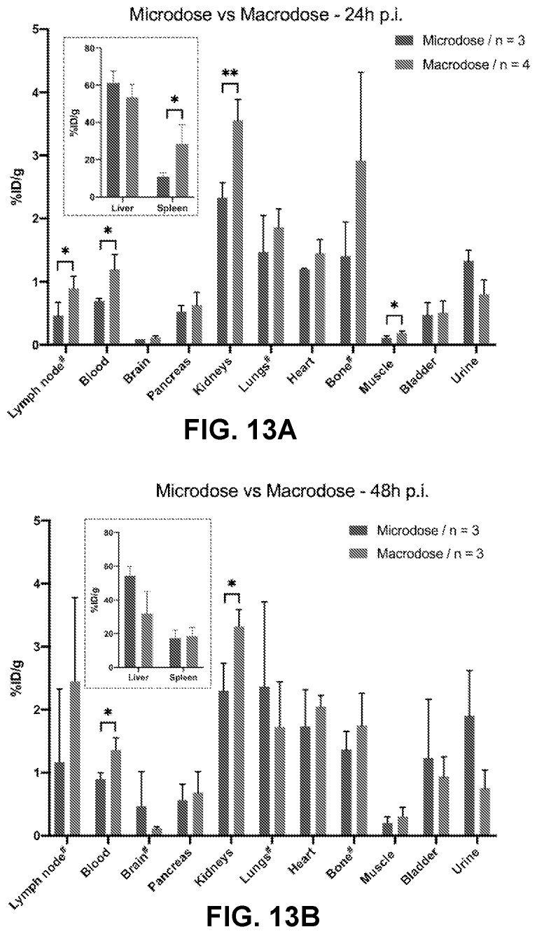

This is also supported by the ex vivo biodistribution analysis indicating that most of the injected dose was present in the liver and the spleen with % ID/g values in the liver of 61.2±6.5 (24 h, microdose), 53.4±7.1 (24 h, macrodose), 54.3±5.5 (48 h, microdose) and 32.1±13.0 (48 h, macrodose) (FIGS. 11A and 11B, see insets). % ID/g values lower than 4 were measured in all other harvested organs and tissues. Because 64 Cu-MN-anti-miR10b accumulates in metastatic lesions, higher % ID/g values were observed in organs bearing metastases such as lymph node, brain, bone, and lung (FIGS. 11A, 11B, 13A, and 13B, #denotes organs with metastatic lesions). Moreover, lymph node metastases were larger in the macrodose group explaining the higher % ID/g values obtained compared to the microdose group. A comparable biodistribution was observed between the microdose and the macrodose at 24 h and 48 h post injection (FIGS. 11A, 11B, 13A, and 13B, note the presence of metastatic lesions in lymph node, brain, lungs, and bone). This is also reflected by the strong correlation with slope near unity that is observed between the % ID/g obtained after administration of a microdose and the % ID/g obtained after administration of a macrodose at 24 h and 48 h p.i. in non-metastatic organs, with a Pearson coefficient of 0.91 (p<0.0001, slope=1.38) and 0.78 (p=0.004, slope=1.05), respectively (FIGS. 11C and 11D).

### Animal Model and Administration of ⁶⁴Cu-MN-Anti-miR10b

Eight-week-old female Balb/c mice (The Jackson Laboratory; Bar Harbor, ME) were implanted orthotopically under the top right third mammary fat pad with the 4T1-Red-Fluc cell line (0.5×10 ⁶ cells). The cells express luciferase and can be detected by non-invasive bioluminescence imaging (BLI) for corresponding analysis of tumor burden. All animals were scanned by BLI to keep track of metastasis formation twice a week. Two weeks after cell inoculation, mice were injected intravenously with ⁶⁴Cu-MN-anti-miR10b. For the microdosing studies, ⁶⁴Cu-MN-anti-miR10b prepared as described above, was injected at a dose of 20 lag as Fe, 118-190 μCi per mouse, n=6. For the carrier-added macrodosing studies, ⁶⁴Cu-MN-anti-miR10b was mixed with NODAGA-MN-anti-miR10b and injected at a dose of 300 μg as Fe, 127-135 μCi per mouse, n=7. An aliquot of the injected dose was analyzed for % ID/g calculations. After PET-MR imaging, mice were sacrificed at 24 hr post injection (n=3 in microdose group and n=4 in macrodose group) and 48 hr post injection (n=3 in each group) for ex vivo biodistribution analysis.

### Bioluminescence Optical Imaging (BLI)

BLI was used to identify metastases. Imaging was performed using the IVIS Spectrum imaging system (Perkin Elmer, Hopkinton, MA). Anesthetized mice were injected intraperitoneally with D-luciferin potassium salt in DPBS (200 mL of 15 mg/mL; Perkin Elmer, Hopkinton, MA) 12 mins before image acquisition. Identical imaging acquisition settings (time, ˜0.5-60 seconds; F-stop, 2; binning, medium) and the same ROI were used to obtain total radiance (photons/sec/cm²/sr) over the whole body. BLI was performed for about 6 to 15 mins to obtain the maximum radiance. All images were processed using the Living Image Software (ver 4.5, IVIS Spectrum, Perkin Elmer, Hopkinton, MA). The total radiance from the bioluminescence readings was used for signal quantification.

### PET-MR Imaging

Mice were imaged in a 4.7 Tesla MRI scanner equipped with a PET insert (Bruker, Billerica MA). Mice were anesthetized with 1-2% isoflurane in medical air. Mice were kept warm using an air heater system and body temperature and respiration rate monitored by a physiological monitoring system (SA Instruments Inc., Stony Brook NY) throughout the imaging session. For the microdosing studies, dynamic PET acquisition was performed continuously for 1 hr after injection of ⁶⁴Cu-MN-anti-miR10b. Mice were then returned to their cages and imaged again at 2 hr, 4 hr, 24 hr and 48 hr post-injection for a period of 30 mins, 30 mins, 60 mins, and 60 mins, respectively. For the macrodosing studies, mice were scanned at 24 hr after injection of ⁶⁴Cu-MN-anti-miR10b for 60 mins. For ex-vivo imaging, organs were positioned onto a plastic holder and scanned for 15 mins.

Anatomic MR images were obtained simultaneously with PET acquisition, including T1-weighted 3D FLASH (Fast Low Angle Shot) sequences with the following parameters: echo time (TE)=3 ms, repetition time (TR)=20 ms, imaging resolution=×0.25×0.5 mm 3/voxel, and flip angle=12 degrees.

PET-MR imaging data were analyzed to estimate the biodistribution and clearance of ⁶⁴Cu-MN-anti-miR10b. Regions of interest (ROIs) were drawn on the MR images over major organs, including heart, liver and kidneys using AMIDE software package (Loening and Gambhir, 2003), and used for quantifying radioactivity for each PET frame. The uptake of ⁶⁴Cu-MN-anti-miR10b in metastases and corresponding tissues without metastases was quantified using ROIs over metastatic bone and lymph node identified by BLI and their non-metastatic contralateral counterparts. Results were expressed as percentage of injected dose per cubic centimeter of tissue (% ID/cc).

### Ex Vivo Biodistribution

Animals were sacrificed at 24 hr and 48 hr post injection. The following organs and tissues were collected: lymph nodes, blood, urine, kidneys, liver, spleen, pancreas, heart, lungs, brain, femur, bladder, and muscle. After resection and ex vivo scanning, organs were weighed and the counts in each organ were measured using a gamma counter (Wizard, Perkin Elmer) with correction for decay.

### Statistical Analysis

Data were expressed as mean±s. d. Statistical comparisons were made using a two-tailed t-test using GraphPad Prism software. A P value of less than 0.05 was considered statistically significant.

### Example 7. PET-MRI of ⁶⁴Cu-MN-Anti-miR10b Accumulation in Tumors and Metastases

Two weeks after orthotopic tumor cell implantation, once metastases were confirmed by BLI, the mice were injected intravenously with ⁶⁴Cu-MN-anti-miR10b. The metastatic organs were identified by in-vivo and ex-vivo BLI. The uptake of ⁶⁴Cu-MN-anti-miR10b by metastases was evaluated by PET-MRI following injection of a no-carrier added microdose (20 μg as Fe, 118-190 μCi per mouse, n=6) or a carrier-added macrodose, as specified above (300 μg as Fe, 127-135 μCi per mouse, n=7). After in vivo PET-MR imaging, the mice were sacrificed at 24 h and 48 h p. i. for ex vivo PET-MR imaging.

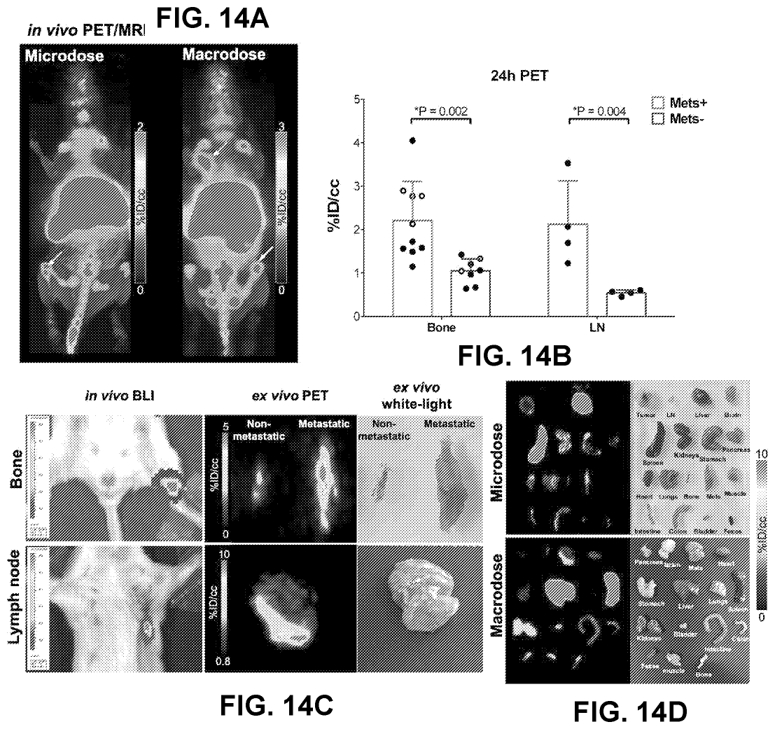

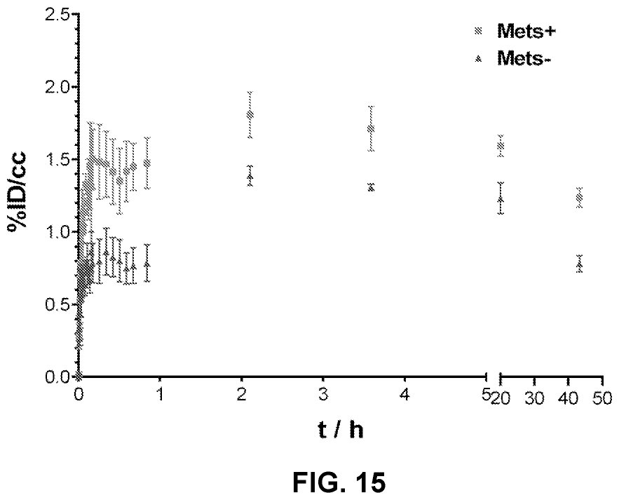

Consistent with the results from the biodistribution studies, the liver and spleen demonstrated a very high PET signal, indicative of hepatic clearance. Renal clearance was the other major clearance pathway as shown by a high PET signal in the kidneys and urine (FIGS. 11A and 11B). Bone and lymph node metastatic lesions could be identified by in vivo PET-MRI, partly because of their spatial separation from the liver and spleen (FIG. 14A). Time-activity curves from metastatic and non-metastatic bones after injection of a microdose of 64 Cu-MN-anti-miR10b showed higher uptake by metastases (FIG. 15). At 24-hr after injection of microdose or macrodose of 64 Cu-MN-anti-miR10b, the metastatic lymph nodes and bone identified by BLI showed significantly higher % ID/cc than the corresponding organs devoid of metastases (FIG. 14B).

BLI images of bone metastatic lesions after injection of a microdose of ⁶⁴Cu-MN-anti-miR10b are shown in FIG. 14C. The uptake of ⁶⁴Cu-MN-anti-miR10b by metastatic lesions was further confirmed by ex vivo PET-MRI (FIG. 14C). The metastatic bone and lymph nodes could be identified by in vivo BLI. These metastatic lesions exhibited higher ex vivo PET signal than their non-metastatic counterparts.

In agreement with ex vivo biodistribution and in vivo PET-MRI, ex vivo imaging showed that the activity associated with the excised liver and spleen was highest in both the macrodosing and microdosing studies. High activity was also seen in organs colonized by tumor cells, such as the lymph nodes, bone, and lungs (FIG. 14D).

Taken together, these observations support a methodology for radiolabeling and imaging of MN-anti-miR10b and other similar nanotherapeutics. These findings point to the feasibility of clinical PET-MRI of therapeutic accumulation in metastatic lesions, as a critical step on the path to translation of related therapeutic agents.

### Discussion

MicroRNA-10b was previously identified as a master regulator of the viability of metastatic tumor cells and designed the therapeutic miR-10b inhibitor, MN-anti-miR10b, for the treatment of metastatic cancer (Ma et al., 2007; Yigit et al., 2013; Yoo et al., 2015). It was demonstrated that MN-anti-miR10b caused complete and persistent regression of local lymph node and distant metastases in breast cancer models with no evidence of systemic toxicity (Yoo et al., 2015; Yoo et al., 2017b).

In this study, key translational experiments were performed that can pave the way for the application of MN-anti-miR10b in patients with advanced metastatic cancer. A radio-labeled derivative of the MN-anti-miR10b therapeutic, 64 Cu-MN-anti-miR10b, was developed. It was demonstrated that radiolabeling the therapeutic did not affect its physico-chemical properties, did not impact cellular uptake in vitro, and preserved effective engagement of its target, based on the complete inhibition of miR10b in tumor cells. It was then shown that microdosing PET-MRI would adequately reflect the biodistribution of a therapeutic dose of the agent and be effective at highlighting metastatic tissues, based on their uptake of ⁶⁴Cu-MN-anti-miR10b.

The tools and methods described herein would enable the demonstration of delivery of the therapeutic to clinical metastases and would enable the clarification of the biodistribution of the agent in cancer patients. Indeed, one of the major challenges facing the development of similar therapeutics lies in the effective delivery to the target organs. In the case of drug delivery to metastases, complicating factors include the larger size of the lesions, as compared to animal models, the heterogeneity of human disease, and differences in the pharmacokinetics of the drugs, due to interspecies hemodynamic variability. Based on these differences, it is not possible to directly extrapolate proof of successful clinical implementation of therapeutic agents from pre-clinical biodistribution and efficacy data.

For that reason, the capacity to carry out microdosing PET studies in patients under an exploratory investigational new drug application protocol represents an important step on the path to clinical approval. Since the PET technique is sensitive enough to determine the concentration of radiolabeled drug with sensitivity approaching the subpicomolar range, as little as a microgram of the radiolabeled drug is generally sufficient to perform the proposed PET study in humans. This characteristic has significant advantages in the initial phases of drug development. Because the low mass of the radiolabeled drug does not induce drug effects, approval from the U.S. Food and Drug Administration for initial human studies may be obtained more quickly and with a more limited preclinical safety and toxicology dossier than is required for therapeutic agents.

In the present study, the fact that the same behavior between the microdose and therapeutic dose was surprisingly observed indicates that it is reasonable to move forward with a microdosing study in patients. The impact of such an exploratory imaging study would be three-fold. First it would establish that MN-anti-miR10b, which is so effective in mice, may also accumulate in human metastases. This greatly de-risks the clinical development of the therapeutic because it shows drug delivery is indeed feasible. Nanotherapeutics have failed because of poor delivery and the fact that human tumors are larger than in mice with different surface-to-volume ratios. Second, the proposed studies may reveal the pharmacokinetic behavior of MN-anti-miR10b which may enable the establishment of dosing during therapy. Third, once MN-anti-miR10b reaches clinical trials, the radiolabeled drug may be used to select patients for treatment, based on which patients'metastases accumulate the therapeutic.

The successful synthesis and testing of ⁶⁴Cu-MN-anti-miR10b is also significant because it sets a precedent for the testing of similar nanotherapeutics based not only the iron oxide delivery platform, as illustrated herein, but also on other nanoparticles that present the possibility of delivering multimodal therapy. For example, copper-based nanomedicine, such as copper cysteamine may be employed to combine RNA-based targeted therapy with copper-cysteamine based X-ray induced photodynamic therapy, which has shown promise in cancer (Li et al., 2010; Ma et al., 2014; Shrestha et al., 2019).

With specific relevance to RNA-based therapeutics, the vast majority of these agents rely on a delivery vehicle, which in many cases comprises a lipid nanoparticle (e.g., Alnylam's Onpattro, Medlmmune's MEDI1191 and AstraZeneca's AZD8601) or GalNAc (e.g., Alnylam's Givlaari, Novartis' Inclisiran, etc.). In some of these cases, it may be possible to employ a related radiolabeling and imaging protocol on the path to full clinical trials, in order to not only de-risk clinical development by demonstrating successful delivery but also to gain further insight into target engagement as a function of dose, schedule, or drug design.

The value of clinical microdose imaging studies may be in answering one of the most key questions on the path to drug development without the associated cost to do a full clinical trial—the question of delivery to the target organ sites. Overall the data described herein show the successful development of a radiolabeled MN-anti-miR10b therapeutic by introducing NODAGA and radioactive ⁶⁴Cu. The biodistribution of ⁶⁴Cu-MN-anti-miR10b was investigated after the injection of a microdose, which showed a comparable biodistribution to the therapeutic macrodose in all organs. The radiolabeling and imaging protocols described herein may advance the clinical development of similar nanotherapeutics by elucidating the pharmacokinetic behavior of the agents, de-risking future clinical trials, and assisting in the selection of patients for treatment, based on which patients' metastases accumulate the therapeutics.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A therapeutic nanoparticle comprising:
a radiolabel;
a chelator that is covalently linked to the therapeutic nanoparticle and to the radiolabel; and
a nucleic acid molecule that is covalently linked to the therapeutic nanoparticle,
wherein the therapeutic nanoparticle has a diameter between about 10 nanometers (nm) to about 30 nm, and
wherein the therapeutic nanoparticle is magnetic.

2. The therapeutic nanoparticle of claim 1, wherein the chelator is covalently-linked to the therapeutic nanoparticle through a chemical moiety comprising a secondary amine.

3. The therapeutic nanoparticle of claim 1, wherein the chelator comprises 1,4,7-triazacyclononane, 1-glutaric acid-4,7-acetic acid (NODAGA).

4. The therapeutic nanoparticle of claim 1, wherein the chelator comprises DOTA, DOTA-GA, p-SCN-Bn-DOTA, CB-TE2A, CB-TE1A1P, AAZTA, MeCOSar, p-SCN-Bn-NOTA, NOTA, HBED-CC, THP, MASS, DFO, or any combination thereof.

5. The therapeutic nanoparticle of claim 1, wherein the radiolabel comprises copper-64 (Cu-64).

6. The therapeutic nanoparticle of claim 1, wherein the radiolabel comprises copper-67 (Cu-67), yttrium-90 (Y-90), terbium-161 (Tb-161), lutetium-177 (Lu-177), bismuth-231(Bi-213), lead-212 (Pb-212), actinium-225 (Ac-225), zirconium-89 (Zr), or any combination thereof.

7. The therapeutic nanoparticle of claim 1, wherein the nucleic acid molecule comprises at least one modified nucleotide.

8. The therapeutic nanoparticle of claim 7, wherein the at least one modified nucleotide is a locked nucleotide.

9. The therapeutic nanoparticle of claim 1, wherein the nucleic acid molecule is an antagomir.

10. The therapeutic nanoparticle of claim 9, wherein the antagomir inhibits microRNA-10b (miR-10b).

11. The therapeutic nanoparticle of claim 1, wherein the nucleic acid molecule is covalently-linked to the nanoparticle through a chemical moiety comprising a disulfide bond.

12. The therapeutic nanoparticle of claim 1, wherein the therapeutic nanoparticle comprises an iron oxide core.

13. The therapeutic nanoparticle of claim 1, wherein the nanoparticle further comprises a polymer coating.

14. The therapeutic nanoparticle of claim 13, wherein the polymer coating comprises dextran.

15. A pharmaceutical composition comprising the therapeutic nanoparticle of claim 1.

16. The pharmaceutical composition of claim 15, further comprising at least one pharmaceutically acceptable carrier or diluent.

17. The pharmaceutical composition of claim 15, wherein the pharmaceutical composition is formulated into a dosage form that is an injectable, a tablet, a lyophilized powder, a suspension, or any combination thereof.

18. A method for decreasing cancer cell invasion or metastasis in a subject having a cancer, the method comprising administering a therapeutic nanoparticle of claim 1 to the subject having the cancer, wherein the therapeutic nanoparticle is administered in an amount sufficient to decrease cancer cell invasion or metastasis in the subject.

19. The method of claim 18, wherein the therapeutic nanoparticle is administered to the subject at a dose that is less than about 0.014 mg/kg.

20. The method of claim 18, wherein the cancer cell metastasis is from a primary tumor to a lymph node in the subject or is from a lymph node to a secondary tissue in a subject.

21-35. (canceled)
